SEQUENCE LISTING (4 sequences)

SEQ ID NO: 1 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 2 - DNA, 1184 bp, Homo sapiens
cacagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatagaaaaaactcgtttgatttttggggggaaacaagggctacctttactggaaaatctggtgatttataaaaaaaaaaaaaaaa

SEQ ID NO: 3 - protein, 114 aa, Homo sapiens
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 4 - DNA, 2475 bp, Homo sapiens
gtgcagaaggcacgaggaagccacagtgctccggatcctccaatcttcgctcctccaatctccgctcctccacccagttcaggaacccgcgaccgctcgcagcgctctcttgaccactatgagcctcctgtccagccgcgcggcccgtgtccccggtccttcgagctccttgtgcgcgctgttggtgctgctgctgctgctgacgcagccagggcccatcgccagcgctggtcctgccgctgctgtgttgagagagctgcgttgcgtttgtttacagaccacgcaaggagttcatcccaaaatgatcagtaatctgcaagtgttcgccataggcccacagtgctccaaggtggaagtggtagcctccctgaagaacgggaaggaaatttgtcttgatccagaagccccttttctaaagaaagtcatccagaaaattttggacggtggaaacaaggaaaactgattaagagaaatgagcacgcatggaaaagtttcccagtcttcagcagagaagttttctggaggtctctgaacccagggaagacaagaaggaaagattttgttgttgtttgtttatttgtttttccagtagttagctttcttcctggattcctcactttgaagagtgtgaggaaaacctatgtttgccgcttaagctttcagctcagctaatgaagtgtttagcatagtacctctgctatttgctgttattttatctgctatgctattgaagttttggcaattgactatagtgtgagccaggaatcactggctgttaatctttcaaagtgtcttgaattgtaggtgactattatatttccaagaaatattccttaagatattaactgagaaggctgtggatttaatgtggaaatgatgtttcataagaattctgttgatggaaatacactgttatcttcacttttataagaaataggaaatattttaatgtttcttggggaatatgttagagaatttccttactcttgattgtgggatactatttaattatttcactttagaaagctgagtgtttcacaccttatctatgtagaatatatttccttattcagaatttctaaaagtttaagttctatgagggctaatatcttatcttcctataattttagacattctttatctttttagtatggcaaactgccatcatttacttttaaactttgattttatatgctatttattaagtattttattaggagtaccataattctggtagctaaatatatattttagatagatgaagaagctagaaaacaggcaaattcctgactgctagtttatatagaaatgtattcttttagtttttaaagtaaaggcaaacttaacaatgacttgtactctgaaagttttggaaacgtattcaaacaatttgaatataaatttatcatttagttataaaaatatatagcgacatcctcgaggccctagcatttctccttggataggggaccagagagagcttggaatgttaaaaacaaaacaaaacaaaaaaaaacaaggagaagttgtccaagggatgtcaattttttatccctctgtatgggttagattttccaaaatcataatttgaagaaggccagcatttatggtagaatatataattatatataaggtggccacgctggggcaagttccctccccactcacagctttggcccctttcacagagtagaacctgggttagaggattgcagaagacgagcggcagcggggagggcagggaagatgcctgtcgggtttttagcacagttcatttcactgggattttgaagcatttctgtctgaatgtaaagcctgttctagtcctggtgggacacactggggttgggggtgggggaagatgcggtaatgaaaccggttagtcagtgttgtcttaatatccttgataatgctgtaaagtttatttttacaaatatttctgtttaagctatttcacctttgtttggaaatccttcccttttaaagagaaaatgtgacacttgtgaaaaggcttgtaggaaagctcctccctttttttctttaaacctttaaatgacaaacctaggtaattaatggttgtgaatttctatttttgctttgtttttaatgaacatttgtctttcagaataggattctgtgataatatttaaatggcaaaaacaaaacataattttgtgcaattaacaaagctactgcaagaaaaataaaacatttcttggtaaaaacgtatgtatttatatattatatatttatatataatatatattatatatttagcattgctgagctttttagatgcctattgtgtatcttttaaaggttttgaccattttgttatgagtaattacatatatattacattcactatattaaaattgtacttttttactatgtgtctcattggttcatagtctttattttgtcctttgaataaacattaaaagatttctaaacttcaaaaaaaaaaaaaaaaaa

(19) United States
(12) Patent Application Publication
(10) Pub. No.: US 2014/0205613 A1
(43) Pub. Date: Jul. 24, 2014

(54) ANTI-TNF AND ANTI-IL 17 COMBINATION THERAPY BIOMARKERS FOR INFLAMMATORY DISEASE

(71) Applicant: AbbVie Inc., Worcester, MA (US)

(72) Inventors: Jeffrey W. Voss, Holden, MA (US); Carolyn A. Cuff, Grafton, MA (US)

(73) Assignee: AbbVie Inc., Worcester, MA (US)

(21) Appl. No.: 14/160,253

(22) Filed: Jan. 21, 2014

Related U.S. Application Data
(60) Provisional application No. 61/754,917, filed on Jan. 21, 2013.

(51) Int. Cl. G01N 33/68; C12Q 1/68; A61K 39/395

(52) U.S. Cl. CPC G01N 33/6863; A61K 39/3955; C12Q 1/6883
USPC 424/158.1; 435/6.13; 435/7.92; 435/6.12; 506/9; 435/6.11

(57) ABSTRACT

The invention provides methods for predicting the efficacy of anti-TNF and anti-IL17 combination therapies in the treatment of a subject suffering from inflammatory disease by determining the level CXCL1 and/or CXCL5 markers in a sample derived from the subject.

### RELATED APPLICATIONS

This application claims priority to U.S. Provisional Application No. 61/754,917, filed on Jan. 21, 2013. The entire contents of the foregoing application are expressly incorporated herein by reference.

### BACKGROUND OF THE INVENTION

Anti-cytokine therapies have become the standard of care for treating the symptoms and arresting the disease progression of inflammatory diseases. But despite the numerous treatment options, many patients still fail to experience a substantial decrease in disease activity. In principle, increasing the level of immunosuppression by combining agents is a plausible strategy for achieving improved efficacy. But attempts to combine anti-cytokine therapies to this end have been plagued by unacceptable safety and tolerability issues (Genovese et al., Arthritis & Rheumatism, 50(5):1412-1419, 2004). Nevertheless, finding the right combination therapy for the treatment of inflammatory disease that can provide both an improved response and acceptable safety remains problematic.

Rheumatoid arthritis (RA) is a chronic systemic autoimmune disease with unknown etiology. Its primary organ manifestations include joint inflammation resulting in pain, swelling and progressive bone and cartilage destruction, with numerous co-morbidities that include anemia and increased risk of cardiovascular events. RA is characterized by infiltration of the synovium by activated lymphocytes, mast cells and neutrophils, resulting in synovial hyperplasia and neovascularization. As of 2012, over 5 million people were afflicted with RA, with approximately 26% having mild, 49% moderate, and 25% severe disease, with women being affected three (3) times more than men. In many cases, current treatment regimens are not completely efficacious.

Anti-TNF therapies are the most prescribed anti-cytokine therapies for RA. TNF is a pro-inflammatory cytokine that increases expression of many mediators of pain, inflammation and joint destruction including chemokines, cytokines, eicosinoids and matrix metalloproteases. In many RA patients that fail to achieve remission, and in rodent disease models, anti-TNF therapy is only partially effective in suppressing the expression of this pro-inflammatory cascade. Based on a number of in vitro studies, TNF appears to cooperate with IL17 in regulating pro-inflammatory gene expression, making the two treatments an attractive candidate for combination therapy. In fact, a recent publication demonstrated increased efficacy of combined anti-TNF/anti-IL17 in mouse CIA (Koenders et al., Arthritis Rheum, 2011, 63(8):2329-2339).

Accordingly, there is a need in the art for inflammatory disease therapies as well as methods for, and compositions useful in, assessing or predicting responsiveness to combined inflammatory disease therapies comprising anti-TNF and anti-IL17.

### SUMMARY OF THE INVENTION

The present invention is based on the identification of novel biomarkers for anti-TNF and anti-IL17 combination therapies. Specifically, the present invention is based, at least in part, on the observation that a combination therapy of an anti-TNF treatment and anti-IL17 treatment can lower a level of expression of a CXCL1 and/or a CXCL5 marker in a subject having an inflammatory disease, relative to a control marker, indicating that the combination therapy is, or will be, effective in treating the subject for the inflammatory disease. Accordingly, the present invention is useful for (i) determining whether a subject will respond to a combination therapy comprising an anti-TNF treatment and anti-IL17 treatment; (ii) monitoring the effectiveness of a combination therapy comprising an anti-TNF treatment and anti-IL17 treatment; (iii) selecting a subject for participation in a clinical trial for a combination therapy comprising an anti-TNF treatment and anti-IL17 treatment; and (iv) identifying a combination therapy comprising an anti-TNF treatment and an anti-IL17 treatment for treating a subject having an inflammatory disease.

Accordingly, in one aspect, the invention provides a method for determining whether a subject having an inflammatory disease will respond to treatment with a combination therapy comprising an anti-TNF treatment and an anti-IL17 treatment. The method includes the steps of determining a level of expression of at least one of a CXCL1 and a CXCL5 marker in a sample obtained from the subject and comparing the level of expression of the marker(s) to the level of expression of a control marker. A higher level of expression of at least one of the CXCL1 and the CXCL5 markers, as compared to the level of expression of the control marker, indicates that the combination therapy will be effective in treating the subject. Alternatively, a lower level of expression of at least one of the CXCL1 and the CXCL5 markers after a combination therapy comprising an anti-TNF treatment and an anti-IL17 treatment, as compared to the level of expression of the control marker, indicates that the combination therapy will be effective in treating the subject.

In another aspect, the present invention provides a method of determining whether a subject having an inflammatory disease will respond to treatment with a combination therapy comprising an anti-TNF treatment and an anti-IL17 treatment. The method includes the steps of processing a sample obtained from the subject such that the sample is transformed, thereby allowing the determination of a level of expression of at least one of a CXCL1 and a CXCL5 marker and comparing the level of expression of the marker(s) to the level of expression of a control marker, e.g., a normal or disease standard or range of laboratory values. A higher level of expression of at least one of the CXCL1 and the CXCL5 markers, as compared to the level of expression of the control marker, indicates that the combination therapy will be effective in treating the subject. Alternatively, a lower level of expression of at least one of the CXCL1 and the CXCL5 markers after a combination therapy comprising an anti-TNF treatment and an anti-IL17 treatment, as compared to the level of expression of the control marker, indicates that the combination therapy will be effective in treating the subject.

In still another aspect, the present invention provides a method of treating a subject having an inflammatory disease with a combination therapy comprising an anti-TNF treatment and an anti-IL17 treatment. The method includes the steps of selecting a subject exhibiting a higher level of expression of at least one of a CXCL1 and a CXCL5 marker as compared to a level of expression of a control marker, e.g., a normal or disease standard or range of laboratory values and administering a therapeutically effective amount of the combination therapy to the subject. Alternatively, a lower level of expression of at least one of the CXCL1 and the CXCL5 markers after a combination therapy comprising an anti-TNF treatment and an anti-IL17 treatment, as compared to the level of expression of the control marker, indicates that the combination therapy will be effective in treating the subject.

In still another aspect, the present invention provides a method of contraindicating a subject having an inflammatory disease from a combination therapy comprising an anti-TNF treatment and an anti-IL17 treatment. The method includes the steps of selecting a subject exhibiting a lower level of expression of at least one of a CXCL1 and a CXCL5 marker as compared to a level of expression of a control marker, e.g., a normal or disease standard or range of laboratory values.

In yet another aspect, the present invention provides a method for monitoring the effectiveness of a treatment with a combination therapy comprising an anti-TNF treatment and an anti-IL17 treatment. The method includes the steps of determining a level of expression of at least one of a CXCL1 and a CXCL5 marker in a sample obtained from a subject following administering a therapeutically effective amount of the combination therapy to the subject and comparing the level of expression of the marker(s) to a level of expression of a control marker, e.g., a normal or disease standard or range of laboratory values. A lower level of expression of at least one of the CXCL1 and the CXCL5 markers, as compared to the level of expression of the control marker, indicates that the combination therapy has been effective in treating the subject.

In another aspect, the present invention provides a method of selecting a subject for participation in a clinical trial for a combination therapy comprising an anti-TNF treatment and an anti-IL17 treatment for the treatment of an inflammatory disease. The method includes the steps of determining a level of expression of at least one of a CXCL1 and a CXCL5 marker in a sample obtained from the subject and comparing the level of expression of the marker(s) to a level of expression of a control marker, e.g., a normal or disease standard or range of laboratory values. A higher level of expression of at least one of the CXCL1 and the CXCL5 markers, as compared to the level of expression of the control marker, indicates that the subject is suitable for participation in the clinical trial. Alternatively, a lower level of expression of at least one of the CXCL1 and the CXCL5 markers after a combination therapy comprising an anti-TNF treatment and an anti-IL17 treatment, as compared to the level of expression of the control marker, indicates that the combination therapy will be effective in treating the subject.

In still another aspect, the present invention provides a method for identifying a combination therapy comprising an anti-TNF treatment and an anti-IL17 treatment suitable for treating a subject having an inflammatory disease. The method includes the steps of determining a level of expression of at least one of the CXCL1 and the CXCL5 marker(s) in a sample obtained from the subject and comparing the level of expression of the marker(s) to a level of expression of a control marker, e.g., a normal or disease standard or range of laboratory values. A higher level of expression of at least one of the CXCL1 and the CXCL5 markers, as compared to the level of expression of the control marker, indicates that the combination therapy will be effective in treating the subject. The method can include testing a plurality of different combination therapies. Alternatively, a lower level of expression of at least one of the CXCL1 and the CXCL5 markers after the combination therapy is administered to the subject, as compared to the level of expression of the control marker, indicates that the combination therapy will be effective in treating the subject.

In yet another aspect, the present invention provides a method of determining whether a subject having an inflammatory disease will respond to treatment with a combination therapy comprising an anti-TNFα antibody and an anti-IL17 antibody. The method includes the steps of determining a level of expression of at least one of a CXCL1 and a CXCL5 marker in a sample obtained from the subject using a reagent that interacts with at least one of the CXCL1 and the CXCL5 marker(s) and transforms the sample such that at least one of the CXCL1 and the CXCL5 marker(s) can be detected and comparing the level of expression of at least one of the CXCL1 and the CXCL5 marker(s) to the level of expression of a control marker. A higher level of expression of at least one of the CXCL1 and the CXCL5 markers, as compared to the level of expression of the control marker, e.g., a normal or disease standard or range of laboratory values, indicates that the combination therapy will be effective in treating the subject. Alternatively, a lower level of expression of at least one of the CXCL1 and the CXCL5 markers after a combination therapy comprising an anti-TNF treatment and an anti-IL17 treatment has been administered, as compared to the level of expression of the control marker, indicates that the combination therapy will be effective in treating the subject.

In still yet another aspect, the present invention provides a kit for (i) determining whether a subject having an inflammatory disease will respond to treatment with a combination therapy comprising an anti-TNF treatment and an anti-IL17 treatment; (ii) monitoring the effectiveness of the combination therapy; (iii) selecting a subject for participation in a clinical trial for the combination therapy; and/or (iv) identifying a combination therapy comprising an anti-TNF treatment and an anti-IL17 treatment for a subject having an inflammatory disease. The kit includes reagents for determining a level of expression of at least one of a CXCL1 and a CXCL5 marker in a sample obtained from the subject and a control marker, e.g., a normal range of values. The kit also includes instructions for (i) determining whether the subject will respond to the combination therapy; (ii) monitoring the effectiveness of the combination therapy; (iii) selecting a subject for participation in a clinical trial for the combination therapy; and/or (iv) identifying a combination therapy comprising an anti-TNF treatment and an anti-IL17 treatment for a subject having an inflammatory disease. Instructions can correspond to any one or more of the aspects described herein.

In one embodiment, any one or more of the aspects described above can be combined with any one or more of the features described below.

In an embodiment, the level of expression of at least one of the CXCL1 and the CXCL5 markers in the sample is determined after a predetermined amount of the anti-TNF treatment and the anti-IL17 treatment are administered to the subject. In an embodiment, the predetermined amount can comprise a sub-therapeutic dose of at least one of the anti-TNF treatment and the anti-IL17 treatment. In another embodiment, the predetermined amount can comprise a sub-therapeutic dose of the anti-TNF treatment and the anti-IL17 treatment. In another embodiment, the predetermined amount can comprise a therapeutic dose of at least one of the anti-TNF treatment and the anti-IL17 treatment. In another embodiment, the predetermined amount can comprise a therapeutic dose of the anti-TNF treatment and the anti-IL17 treatment.

In an embodiment, the level of expression of the control marker is the level of expression of the control marker in the sample before a predetermined amount of the anti-TNF treatment and the anti-IL17 treatment are administered to the subject.

In an embodiment, the level of expression of the control marker is an average level of expression of the control marker in a population of subjects suffering from the inflammatory disease. In another embodiment, the level of expression of the control marker is the level of expression of the marker in the subject before combination therapy with an anti-TNF treatment and an anti-IL17 treatment.

In an embodiment, the control marker comprises a CXCL1 marker or a CXCL5 marker. In another embodiment, the control marker comprises both a CXCL1 marker and a CXCL5 marker.

In an embodiment, the population of subjects suffering from the inflammatory disease has received at least one of the anti-TNF treatment and the anti-IL17 treatment. In one embodiment, the population of subjects suffering from the inflammatory disease has received the anti-TNF treatment and the anti-IL17 treatment.

In an embodiment, the anti-TNF treatment comprises an anti-TNF binding protein. In an embodiment, the anti-TNF binding protein comprises an antibody, or antigen binding fragment thereof, that specifically binds to the protein. In another embodiment, the anti-TNF antibody, or antigen binding fragment thereof, is a murine antibody, a human antibody, a humanized antibody, a bispecific antibody, a chimeric antibody, a Fab, a Fab′, a F(ab′)₂, an scFv, an SMIP, an affibody, an avimer, a versabody, a nanobody, a domain antibody, or an antigen binding fragment of any of the foregoing.

In an embodiment, the anti-TNF antibody is an anti-TNFα antibody, e.g., a human anti-TNF antibody (e.g., Adalimumab®, or an antigen binding fragment thereof). In another embodiment, the anti-TNF antibody comprises a humanized anti-TNF antibody, (e.g., infliximab, or an antigen binding fragment thereof).

In an embodiment, the anti-TNFα binding protein comprises a fusion protein (e.g., etanercept, or an antigen binding fragment thereof).

In an embodiment, the anti-IL17 treatment comprises an anti-IL17 binding protein. In an embodiment, the anti-IL17 binding protein comprises an antibody, or antigen binding fragment thereof, that specifically binds to the protein. In another embodiment, the anti-IL17 antibody, or antigen binding fragment thereof, is a murine antibody, a human antibody, a humanized antibody, a bispecific antibody, a chimeric antibody, a Fab, a Fab′, a F(ab′)₂, an ScFv, an SMIP, an affibody, an avimer, a versabody, a nanobody, a domain antibody, or an antigen binding fragment of any of the foregoing.

In an embodiment, the anti-IL17 antibody comprises a human antibody (e.g., secukinumab or RG7624, or an antigen binding fragment thereof). In an embodiment, the anti-IL17 antibody comprises a humanized antibody (e.g., 10F7, B6-17, or an antigen binding fragment thereof).

In an embodiment, the anti-IL17 binding protein comprises a fusion protein.

In an embodiment, the anti-TNF treatment comprises methotrexate, an analog thereof, or a pharmaceutically acceptable salt thereof. In an embodiment, the anti-IL17 treatment comprises methotrexate, an analog thereof, or a pharmaceutically acceptable salt thereof. In an embodiment, at least one of the anti-TNF treatment and the anti-IL17 treatment comprises methotrexate, an analog thereof, or a pharmaceutically acceptable salt thereof. In an embodiment, both the anti-TNF treatment and the anti-IL17 treatment comprise methotrexate, an analog thereof, or a pharmaceutically acceptable salt thereof.

In an embodiment, the combination therapy comprises the administration of a multispecific binding protein that binds at least one of TNF and IL17. In an embodiment, the combination therapy comprises the administration of a multispecific binding protein that binds TNF and IL17. In an embodiment, the multispecific binding protein comprises a dual variable domain immunoglobulin (DVD-Ig™) molecule, a half-body DVD-Ig (hDVD-Ig) molecule, a triple variable domain immunoglobulin (tDVD-Ig) molecule, a receptor variable domain immunoglobulin (rDVD-Ig) molecule, a polyvalent DVD-Ig (pDVD-Ig) molecule, a monobody DVD-Ig (mDVD-Ig) molecule, a cross over (coDVD-Ig) molecule, a blood brain bather (bbbDVD-Ig) molecule, a cleavable linker DVD-Ig (clDVD-Ig) molecule, or a redirected cytotoxicity DVD-Ig (rcDVD-Ig) molecule that binds at least one of TNF and IL17.

In an embodiment, the level of expression of at least one of the CXCL1 and the CXCL5 markers is determined. In another embodiment, the level of expression of the CXCL1 and the CXCL5 markers is determined. In one example, a higher level of expression of at least one of the CXCL1 and the CXCL5 markers as compared to the level of expression of the control marker indicates that the combination therapy will be effective. In another example, a higher level of expression of both the CXCL1 and the CXCL5 markers as compared to the level of expression of the control marker indicates that the combination therapy will be effective. In one example, a lower level of expression of at least one of the CXCL1 and the CXCL5 markers as compared to the level of expression of the control marker indicates that the combination therapy is effective. In another example, a lower level of expression of both the CXCL1 and the CXCL5 marker as compared to the level of expression of the control marker indicates that the combination therapy is effective.

In an embodiment, the subject has not been previously treated with a monotherapy comprising an anti-TNF treatment or a monotherapy comprising an anti-IL17 treatment.

In an embodiment, the combination therapy decreases the level of expression of at least one of the CXCL1 and the CXCL5 markers to a greater extent than a monotherapy comprising an anti-TNF treatment. In an embodiment, the combination therapy decreases the level of expression of the CXCL1 and the CXCL5 markers to a greater extent than a monotherapy comprising an anti-TNF treatment.

In an embodiment, the combination therapy has a better clinical outcome or clinical endpoint than a monotherapy comprising an anti-TNF treatment.

In an embodiment, the subject does not respond to a monotherapy comprising an anti-TNF treatment.

In an embodiment, the combination therapy decreases the level of expression of at least one of the CXCL1 and the CXCL5 markers to a greater extent than a monotherapy comprising an anti-IL17 treatment.

In an embodiment, the combination therapy has a better clinical outcome or clinical endpoint than a monotherapy comprising an anti-IL17 treatment.

In an embodiment, the subject does not respond to a monotherapy comprising an anti-IL17 treatment.

In an embodiment, the combination therapy decreases the level of expression of at least one of the CXCL1 and the CXCL5 markers to a greater extent than both a monotherapy comprising an anti-TNF treatment and a monotherapy comprising an anti-IL17 treatment.

In an embodiment, the combination therapy has a better clinical outcome or clinical endpoint than both a monotherapy comprising an anti-TNF treatment and a monotherapy comprising an anti-IL17 treatment.

In an embodiment, the subject does not respond to either a monotherapy comprising an anti-TNF treatment or a monotherapy comprising an anti-IL17 treatment.

In an embodiment, the level of expression of the CXCL1 and/or the CXCL5 marker(s) is determined at the nucleic acid level. In an embodiment, the level of expression of the CXCL1 and/or the CXCL5 marker(s) can be determined by detecting RNA, e.g., mRNA, miRNA, or hnRNA. In another embodiment, the level of expression of the CXCL1 and/or the CXCL5 marker(s) is determined by detecting DNA (e.g., cDNA). In an embodiment, the level of expression of the CXCL1 and/or the CXCL5 marker(s) may be determined by using a polymerase chain reaction (PCR) amplification reaction, reverse-transcriptase PCR analysis, quantitative reverse-transcriptase PCR analysis, Northern blot analysis, an RNAase protection assay, digital RNA detection/quantitation, and combinations or sub-combinations thereof.

In an embodiment, the CXCL1 and/or the CXCL5 marker(s) comprise a protein. The protein can be detected using a binding protein that binds at least one of the CXCL1 and the CXCL5 markers. In one embodiment, the binding protein is an antibody, or antigen-binding portion thereof, that binds at least one of the CXCL1 and the CXCL5 markers. In one embodiment, the antibody is an anti-CXCL1 antibody, or antigen-binding portion thereof, that specifically binds to CXCL1 and/or an anti-CXCL5 antibody, or antigen-binding portion thereof, that specifically binds to CXCL5. In one embodiment, the antibody is an antibody, or antigen-binding portion thereof, that specifically binds to CXCL1 and CXCL5.

In an embodiment, the antibody or antigen binding fragment thereof comprises a label, e.g., a radio-label, a biotin label, a chromophore, a fluorophore, and an enzyme.

In an embodiment, the level of expression of at least one of the CXCL1 and the CXCL5 markers is determined by using an immunoassay, a western blot analysis, a radioimmunoassay, immunofluorimetry, immunoprecipitation, equilibrium dialysis, immunodiffusion, an electrochemiluminescence immunoassay (ECLIA), an ELISA assay, immunopolymerase chain reaction, or a combination or sub-combination thereof. In an embodiment, the immunoassay comprises a solution-based immunoassay, e.g., comprising electrochemiluminescence, chemiluminescence, fluorogenic chemiluminescence, fluorescence polarization, or time-resolved fluorescence. In an embodiment, the immunoassay comprises a sandwich immunoassay, e.g., comprising electrochemiluminescence, chemiluminescence, or fluorogenic chemiluminescence.

In an embodiment, the level of expression of the CXCL1 and/or the CXCL5 marker in the sample is determined in vitro.

In an embodiment, the level of expression of at least one of the CXCL1 and the CXCL5 markers is determined by using a bioassay, e.g., an ex vivo assay where a patient's cells (e.g., monocytes) are removed and tested in culture with the combination therapy.

In an embodiment, the sample comprises a fluid, or component thereof, obtained from the subject. In an embodiment, the fluid comprises at least one of amniotic fluid, aqueous humor, vitreous humor, bile, blood, breast milk, cerebrospinal fluid, cerumen, chyle, cystic fluid, endolymph, feces, gastric acid, gastric juice, lymph, mucus, nipple aspirates, pericardial fluid, perilymph, peritoneal fluid, plasma, pleural fluid, pus, saliva, sebum, semen, sweat, serum, sputum, synovial fluid, tears, urine, vaginal secretions, and fluid collected from a biopsy.

In an embodiment, the sample comprises a tissue or cell, or component thereof, obtained from the subject.

In an embodiment, the sample is from a human subject who exhibits at least one symptom of an inflammatory disease. In one embodiment, the sample is from a human subject who exhibits at least one symptom of rheumatoid arthritis. Symptoms of rheumatoid arthritis include, but are not limited to, swollen joints, painful joints, inflammation and/or bone loss. In one embodiment, the sample is from a human subject who exhibits at least one symptom of psoriasis (which may include, but are not limited to, skin inflammation, skin irritation, skin redness, skin lesions, nail pitting, nail separation, nail thickening and/or nail discoloration), psoriatic arthritis (which may include, but are not limited to, arthritis of the fingers, arthritis of the spine, arthritis mutilans and/or bone erosion associated with psoriasis), ankylosing spondylitis (which may include, but are not limited to, scroilitis, clerosis, inflammation of one or more vertebrae, inflammation of sacroiliac joints, and/or inflammation of joints between the spine or pelvis), juvenile idiopathic arthritis (which may include, but are not limited to, joint pain, joint swelling, joint stiffness, trouble sleeping, problems walking and/or fever and rash), Behcet's disease (which may include, but are not limited to, mouth sores, skin lesions, genital sores or lesions, uveitis, joint pain and/or swelling, inflammation in veins and arteries, vasculitis, abdominal pain, diarrhea and/or inflammation in the brain and/or nervous system), spondyloarthritis (which may include, but are not limited to, back pain, pain and swelling in the arms and/or legs and/or spinal fusion), uveitis (which may include, but are not limited to, swelling of the uvea, blurred vision, eye redness, eye irritation, eye pain and/or floating spots in the vision) or systemic lupus erythematosus (which may include, but are not limited to, fatigue, joint and muscle pain, skin rashes, sensitivity to light, pericarditis and/or pleurisy).

In an embodiment, the subject is a human subject. In one embodiment, the subject has an inflammatory disease. In one embodiment, the inflammatory disease is rheumatoid arthritis. In another embodiment, the inflammatory disease is psoriasis, psoriatic arthritis, ankylosing spondylitis, juvenile idiopathic arthritis, Behcet's disease, spondyloarthritis, uveitis or systemic lupus erythematosus (SLE).

In an embodiment, the combination therapy comprises a DVD-Ig™ molecule directed against TNF and IL17. In an embodiment, the DVD-Ig™ molecule binds TNFα and IL17.

In an embodiment, the reagent that interacts with at least one of the CXCL1 and the CXCL5 markers is an anti-CXCL1 or an anti-CXCL5 antibody, or antigen binding fragment thereof. In one embodiment, the antibody, or antigen-binding portion thereof, specifically binds to CXCL1 and/or CXCL5. In one embodiment, the method comprises processing a sample from the subject and performing a binding assay comprising contacting the processed sample with an antibody to CXCL1 and/or CXCL5 to form a complex between the antibody and CXCL1 and/or CXCL5 present in the sample, and detecting the formation of a complex.

Exemplary anti-CXCL1 antibodies include, but are not limited to, EMD Millipore: AP1151-100UG; Everest Biotech: EB09637; Lifespan Biosciences: LS-B2843, LS-B2513, and LS-C108147; eBioscience: 50-7519-42 and 50-7519-41; AbD Serotec: AAM40B, AAM40, and AAR22B; Thermo Fisher Scientific, Inc.: PA1-32959, PA1-32924, and PA1-20861; Abbiotec: 251349, 12335-1-AP, and AP08852PU-N; NovaTeinBio: 63059; Abgent: AT1688a; Aviva Systems Biology: AVARP07032_P050, OASA08635, and OAEB00281; United States Biological: C8297-97A, C8298-01B, and C8298-01C; Creative Biomart: CAB-1005 MH, CAB-3086 MH, and CAB-115 MH; Novus Biologicals: NBP1-61297, NBP1-51486, and NBP1-19301; Abnova: H00002919-M01, H00002919-D01P, and H00002919-M03; and Fitzgerald: 70R-10502; and ProSci: 31-057, 42-129, and 42-196.

Exemplary anti-CXCL5 antibodies include Lifespan Biosciences: LS-B5529 and AbD Serotec: AHP1279, AAM42, and AHP1279B; Proteintech Group: 10809-1-AP and PA1-29657; Biorbyt: orb13909 and orb13450; Acris Antibodies: AM31037PU-N, PP1003B2, and PP1003P1; NovaTeinBio: 63066, AT1694a, and AT1693a; Aiva Systems Biology: OASA07658, OASA08449 and OASA07657; United States Biological: C8297-98H1, C8297-98H, and E2275-07; Creative Biomart: CAB-5426 MH and CAB-5425 MH; Novus Biologicals: 33140002; and Abnova: H00006374-M05, H00006374-M03, and H00006374-B01.

In an embodiment, the reagent that interacts with at least one of the CXCL1 and CXCL5 markers comprises a nucleic acid probe specific for at least one of the CXCL1 and CXCL5 marker(s). In one embodiment, the method comprises processing a sample from the subject and performing a binding assay comprising contacting the processed sample with a probe to CXCL1 and/or CXCL5 to form a complex between the probe and CXCL1 and/or CXCL5 present in the sample, and detecting the formation of the complex.

In an embodiment, determining the level of expression of at least one of the CXCL1 and CXCL5 markers in the sample comprises performing an immunoassay using an anti-CXCL1 or an anti-CXCL5 antibody. In an embodiment, determining the level of expression of at least one of the CXCL1 and the CXCL5 markers in the sample comprises performing an immunoassay using an anti-CXCL1 and an anti-CXCL5 antibody.

In an embodiment, determining the level of expression of at least one of the CXCL1 and CXCL5 markers in the sample comprises a novel combination of assays.

In an embodiment, the inflammatory disease comprises rheumatoid arthritis. In other embodiments, the inflammatory disease comprises at least one of psoriasis, psoriatic arthritis, ankylosing spondylitis, juvenile idiopathic arthritis, Behcet's disease, spondyloarthritis, uveitis, and systemic lupus erythematosus.

In one embodiment, the methods of determining whether a subject having an inflammatory disease will respond to treatment with a combination therapy comprising an anti-TNF treatment and an anti-IL17 treatment further comprise the step of administering to the patient the combination therapy comprising the anti-TNF treatment and the anti-IL17 treatment.

In an embodiment, the marker comprises a gene product. The gene product can comprise a protein or RNA.

In still yet another aspect, the present invention provides a kit for (i) determining whether a subject having an inflammatory disease will respond to treatment with a combination therapy comprising an anti-TNF treatment and an anti-IL17 treatment; (ii) monitoring the effectiveness of the combination therapy; (iii) selecting a subject for participation in a clinical trial for the combination therapy; and/or (iv) identifying a combination therapy comprising an anti-TNF treatment and an anti-IL17 treatment for a subject having an inflammatory disease. The kit includes reagents for determining a level of expression of at least one of a CXCL1 and a CXCL5 marker in a sample obtained from the subject and a control marker. The kit also includes instructions for (i) determining whether the subject will respond to the combination therapy; (ii) monitoring the effectiveness of the combination therapy; (iii) selecting a subject for participation in a clinical trial for the combination therapy; and/or (iv) identifying a combination therapy comprising an anti-TNF treatment and an anti-IL17 treatment for a subject having an inflammatory disease. Instructions can correspond to any one or more of the aspects described herein.

In an embodiment, the kit's reagents for determining the level of expression of at least one of a CXCL1 and a CXCL5 marker comprise a probe for amplifying and detecting at least one of the CXCL1 and the CXCL5 markers.

In an embodiment, the kit's reagents for determining the level of expression of at least one of a CXCL1 and a CXCL5 marker comprise an antibody, or antigen binding fragment thereof.

In an embodiment, the kit further comprises reagents for obtaining a biological sample from the subject.

### BRIEF DESCRIPTION OF THE DRAWINGS

FIG. 1A shows the amino acid sequence for the human CXCL1 protein.

FIG. 1B shows the nucleic acid sequence for the human CXCL1 gene.

FIG. 2A shows the amino acid sequence for the human CXCL5 protein.

FIG. 2B shows the nucleic acid sequence for the human CXCL5 gene.

FIG. 3 shows that a combination therapy of an anti-TNF treatment and an anti-IL-17 treatment confers superior protection in a mouse collagen induced arthritis (CIA) model compared to an anti-TNF treatment alone or an anti-IL-17 treatment alone.

FIG. 4 shows that a combined anti-TNF and anti-IL-17 treatment demonstrates superior bone protection compared to an anti-TNF treatment alone or an anti-IL17 treatment alone.

FIG. 5 shows that IL17 and TNF synergistically up-regulate gene expression of the CXCL1 and CXCL5 genes in both mouse and human synoviocytes.

FIG. 6 shows that IL17 and TNF synergistically up-regulate CXCL1 and CXCL5 genes in mouse chondrocyte cells.

FIG. 7 shows CXCL1 and CXCL5 protein levels in paw lysates and serum of CIA mice treated with anti-TNF antibody alone, anti-IL17 antibody alone, and anti-TNF antibody and anti-IL17 antibody in combination.

FIG. 8 shows upregulation of CXCL1 and CXCL5 in RA patients as compared to normal control.

FIG. 9 shows that there is no significant difference between the CXCL1 and CXCL5 levels in RA patients treated with anti-TNF vs. RA patients treated with anti-TNF+methotrexate and that these patients were insensitive to either monotreatment.

FIG. 10 shows the numerical results of the experiment depicted graphically in FIG. 9.

### DETAILED DESCRIPTION OF THE INVENTION

The present invention is based on the identification of novel biomarkers for anti-TNF and anti-IL17 combination therapies. Specifically, the present invention is based, at least in part, on the observation that a combination therapy of an anti-TNF treatment and an anti-IL17 treatment lowers the level of expression of a CXCL1 and/or a CXCL5 marker in a subject having an inflammatory disease, relative to a control marker, indicating that the combination therapy is, or will be, effective in treating the subject for the inflammatory disease. Accordingly, the present invention is useful for (i) determining whether a subject will respond to a combination therapy comprising an anti-TNF treatment and an anti-IL17 treatment; (ii) monitoring the effectiveness of a combination therapy comprising an anti-TNF treatment and an anti-IL17 treatment; (iii) selecting a subject for participation in a clinical trial for a combination therapy comprising an anti-TNF treatment and an anti-IL17 treatment; and/or (iv) identifying a combination therapy comprising an anti-TNF treatment and an anti-IL17 treatment for treating a subject having an inflammatory disease.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms, e.g., those characterized by “a” or “an”, shall include pluralities, e.g., one or more markers (e.g., biomarkers); “some”, “certain”, and “various”. In this application, the use of “or” means “and/or”, unless stated or differentiated otherwise. Furthermore, the use of the terms “including” and “comprising,” as well as other forms of the terms, such as “includes”, “included”, “comprises”, and “comprised of”, are not limiting. Also, terms such as “element” or “component” encompass both elements and components comprising one unit and elements and components that comprise more than one unit unless specifically stated otherwise.

The phrase “determining whether a subject having an inflammatory disease will respond to treatment with a combination therapy comprising an anti-TNF treatment and an anti-IL17 treatment” refers to assessing the likelihood that treatment of a subject with a dose of the combination therapy will be therapeutically effective (e.g., provide a therapeutic benefit to the subject) or will not be therapeutically effective in the subject. Assessment of the likelihood that treatment will or will not be therapeutically effective typically can be performed before treatment has begun or before treatment is resumed. Alternatively or in combination, assessment of the likelihood of effective treatment can be performed during treatment, e.g., to determine whether treatment should be continued or discontinued.

The term “anti-TNF treatment” includes any treatment for a TNF associated disease and/or any treatment that affects (e.g., inhibits) the TNF pathway. This term includes TNF antagonists that have the effect of binding to or neutralizing, inhibiting, reducing, or negatively modulating the activity of tumor necrosis factor (TNF). In an embodiment, the anti-TNF treatment comprises an anti-TNF binding protein. In an embodiment, the anti-TNF treatment can comprise an anti-TNF antibody, or an antigen binding fragment thereof. In an embodiment, an antibody is a murine antibody, a human antibody, a humanized antibody, a bispecific antibody, a chimeric antibody, a Fab, a Fab′, a F(ab′)₂, an ScFv, an SMIP, an affibody, an avimer, a versabody, a nanobody, a domain antibody, and an antigen binding fragment of any of the foregoing.

In an embodiment, the anti-TNF antibody comprises an anti-TNFα antibody, e.g., a human anti-TNF antibody, e.g., a human anti-TNFα antibody, e.g., Adalimumab®, or an antigen binding fragment thereof (see U.S. Pat. No. 6,090,382). In another embodiment, the anti-TNF antibody comprises a humanized anti-TNF antibody, e.g., infliximab, or an antigen binding fragment thereof. In another embodiment, the anti-TNF binding protein comprises a fusion protein, e.g., etanercept, or an antigen binding fragment thereof. In other embodiments, the anti-TNF treatment comprises methotrexate, an analog thereof, or a pharmaceutically acceptable salt thereof.

In an embodiment, the anti-TNF comprises a multispecific binding protein. In an embodiment, the multispecific binding protein comprises a dual variable domain immunoglobulin (DVD-Ig™) molecule, a half-body DVD-Ig (hDVD-Ig) molecule, a triple variable domain immunoglobulin (tDVD-Ig) molecule, a receptor variable domain immunoglobulin (rDVD-Ig) molecule, a polyvalent DVD-Ig (pDVD-Ig) molecule, a monobody DVD-Ig (mDVD-Ig) molecule, a cross over (coDVD-Ig) molecule, a blood brain barrier (bbbDVD-Ig) molecule, a cleavable linker DVD-Ig (clDVD-Ig) molecule, or a redirected cytotoxicity DVD-Ig (rcDVD-Ig) molecule.

The term“anti-IL17 treatment” includes any treatment for an IL17 associated disease and/or any treatment that affects (e.g., inhibits) the IL17 pathway. This term includes IL17 antagonists that have the effect of binding to or neutralizing, inhibiting, reducing, or negatively modulating the activity of interleukin 17 (IL17). In an embodiment, the anti-IL17 treatment comprises an anti-IL17 binding protein. In another example, the anti-IL17 binding protein comprises a fusion protein. In an embodiment, the anti-IL17 treatment comprises an anti-IL17 antibody, or an antigen binding fragment thereof. In an embodiment, the anti-IL17 antibody comprises a human antibody, e.g., secukinumab and RG7624, or an antigen binding fragment thereof. In an embodiment, the anti-IL17 antibody comprises a humanized antibody, for example ixekizumab, 10F7, B6-17, or an antigen binding fragment thereof. In other embodiments, the anti-IL17 treatment comprises methotrexate, an analog thereof, or a pharmaceutically acceptable salt thereof. In an embodiment, the anti-IL17 can include a multispecific binding protein, as described above and, in more detail, below.

Antibodies used in immunoassays to determine the level of expression of the biomarker, may be labeled with a detectable label. The term “labeled”, with regard to the probe or antibody, is intended to encompass direct labeling of the probe or antibody by incorporation of a label (e.g., a radioactive atom), coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In one embodiment, the antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair (e.g., biotin-streptavidin), or an antibody fragment (e.g., a single-chain antibody, or an isolated antibody hypervariable domain) which binds specifically with the biomarker is used.

The phrase “inflammatory disease” refers to a disease or disorder characterized by chronic or acute inflammation. Numerous inflammatory diseases are known in the art, such as arthritis, including rheumatoid arthritis, osteoarthritis, psoriatic arthritis, juvenile idiopathic arthritis; necrotizing enterocolitis (NEC); gastroenteritis; intestinal flu; stomach flu; pelvic inflammatory disease (PID); emphysema; pleurisy; pyelitis; pharyngitis; sore throat; angina; acne vulgaris; rubor; urinary tract infection; appendicitis; bursitis; colitis; cystitis; dermatitis; phlebitis; rhinitis; tendonitis; tonsillitis; vasculitis; asthma; autoimmune diseases; celiac disease; chronic prostatitis; glomerulonephritis; hypersensitivities; inflammatory bowel diseases; pelvic inflammatory disease; reperfusion injury; sarcoidosis; transplant rejection; vasculitis; interstitial cystitis; hay fever; periodontitis; atherosclerosis; psoriasis; ankylosing spondylitis; juvenile idiopathic arthritis; Behcet's disease; spondyloarthritis; uveitis; systemic lupus erythematosus, and some cancers (e.g., gallbladder carcinoma).

The terms “marker” or “biomarker” are used interchangeably herein to mean a substance that is used as an indicator of a biologic state, e.g., genes, messenger RNAs (mRNAs, microRNAs (miRNAs)); heterogeneous nuclear RNAs (hnRNAs), and proteins, or portions thereof.

The “level of expression” or “expression pattern” refers to a quantitative or qualitative summary of the expression of one or more markers or biomarkers in a subject, such as in comparison to a standard or a control.

A “higher level of expression” or an “increase in the level of expression” of CXCL1 and/or CXCL5 refers to an expression level in a test sample that is greater than the standard error of the assay employed to assess expression, and is preferably at least twice, and more preferably three, four, five, six, seven, eight, nine, or ten or more times the expression level of CXCL1 and/or CXCL5 in a control sample (e.g., a sample from a healthy subject not afflicted with inflammatory disease, e.g., RA, and/or a sample from a subject(s) having slow disease progression and/or, the average expression level of CXCL1 and/or CXCL5 in several control samples).

A “lower level of expression” or a “decrease in the level of expression” of CXCL1 and/or CXCL5 refers to an expression level in a test sample that is less than the standard error of the assay employed to assess expression, and preferably at least twice, and more preferably three, four, five, six, seven, eight, nine, or ten or more times less than the expression level of CXCL1 and/or CXCL5 in a control sample (e.g., a sample from a subject with rapid disease progression and/or a sample from the subject prior to administration of a portion of a therapy for inflammatory disease, e.g., RA, and/or the average expression level of CXCL1 and/or CXCL5 in several control samples).

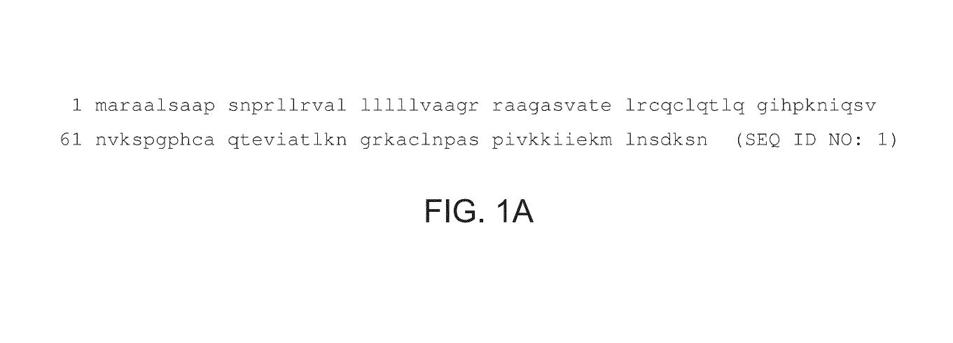

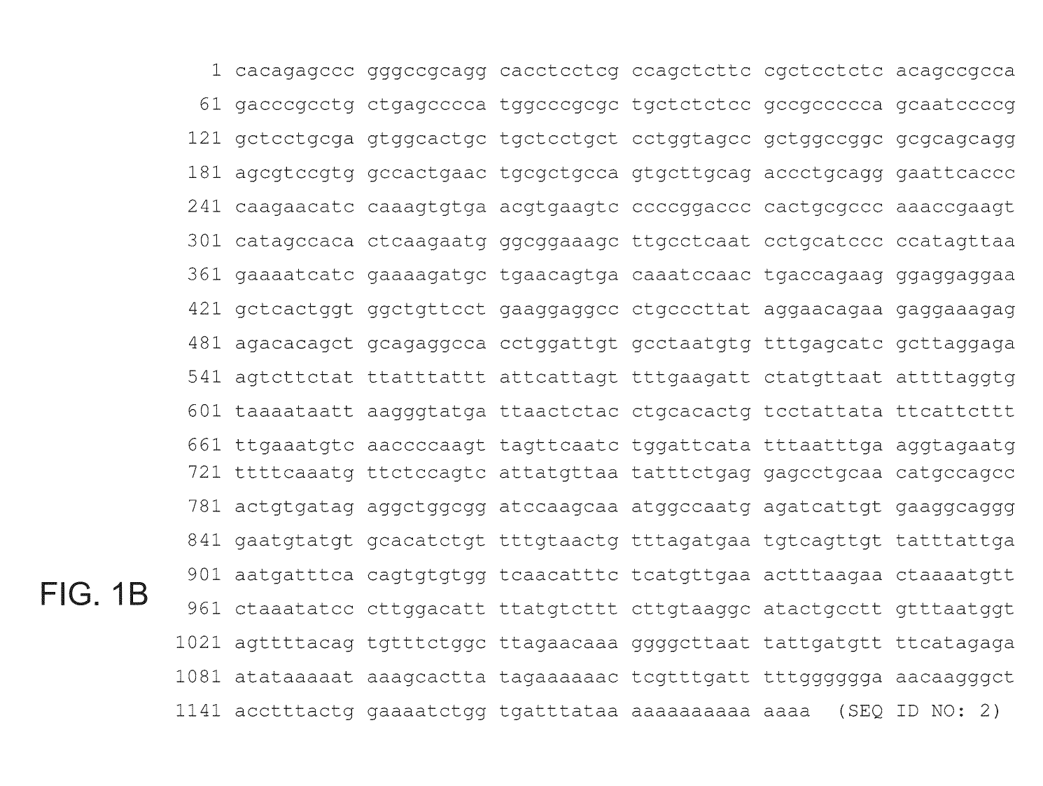

The term “CXCL1” refers to the gene for chemokine ligand 1, which is a small cytokine belonging to the CXC chemokine family that was previously called GRO1 oncogene, GROα, KC, Neutrophil-activating protein 3 (NAP-3) and melanoma growth stimulating activity alpha (MSGA-α). In humans, this protein is encoded by the CXCL1 gene. In other animals, this protein is encoded by orthologous genes. The nucleotide and amino acid sequences of CXCL1 are known in the art and can be found for example, in publically available databases such as the NCBI GenBank. The human CXCL1 gene can be found under GenBank Accession No. AAH11976.1 and the human CXCL1 protein can be found under NCBI Reference Sequence NM_{—}001511.3. The sequences of the human CXCL1 protein and gene can be found in FIGS. 1A and 1B.

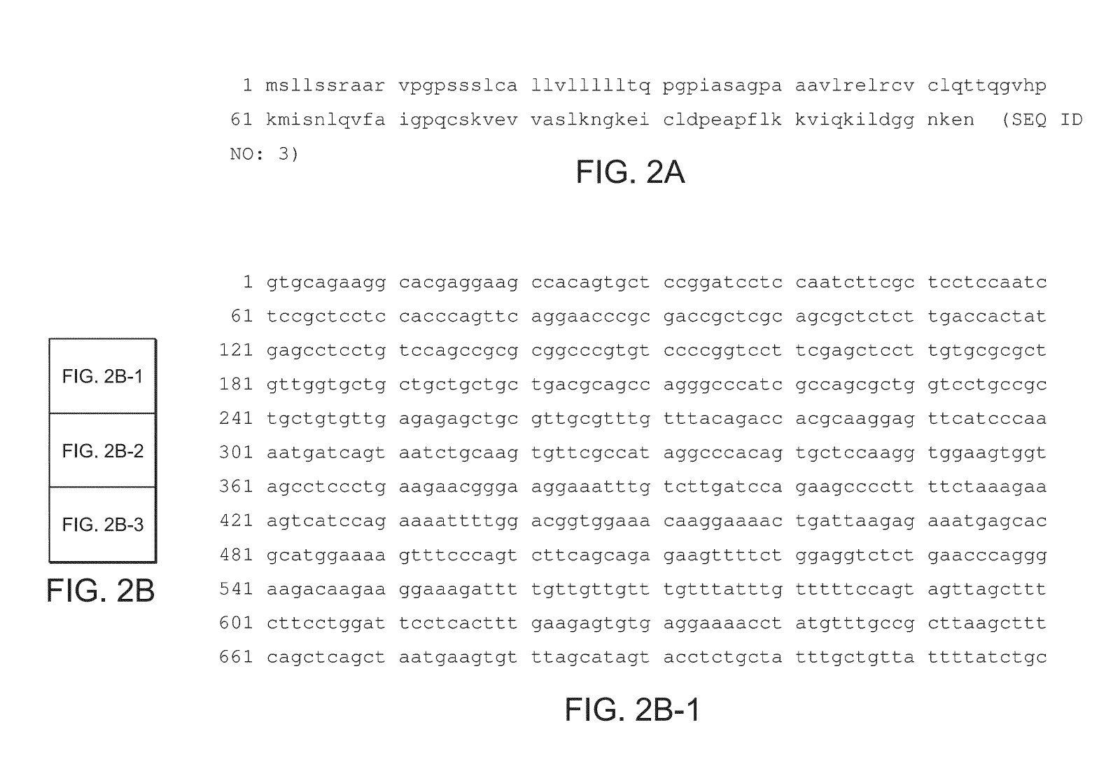

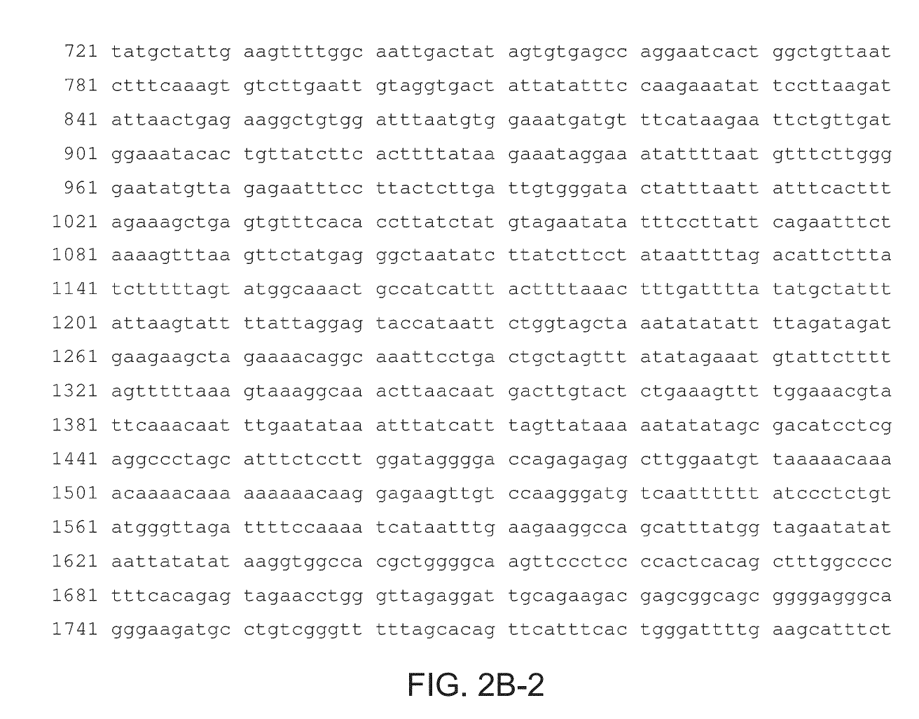

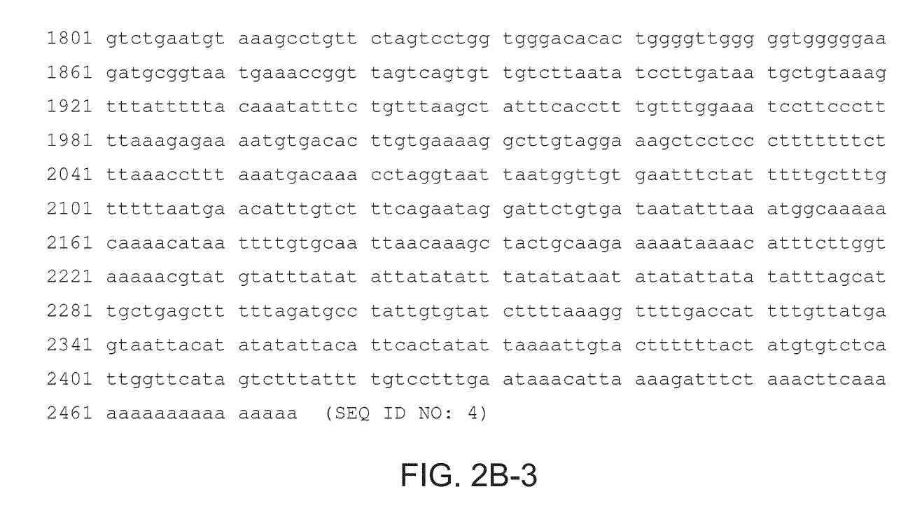

The term “CXCL5” refers to the gene for CXCL5, which is a small cytokine belonging to the CXC chemokine family that is also known as epithelial-derived neutrophil-activating peptide 78 (ENA-78). CXCL5 is produced following stimulation of cells with the inflammatory cytokines interleukin-1 or tumor necrosis factor-alpha. In humans, this protein is encoded by the CXCL5 gene. In other animals, this protein is encoded by orthologous genes. The nucleotide and amino acid sequences of CXCL5 are known in the art and can be found for example, in publically available databases such as the NCBI GenBank. The human CXCL1 gene can be found under GenBank Accession No. EAX05696.1 and the human CXCL1 protein can be found under NCBI Reference Sequence NM_{—}002994.3. The sequences of the human CXCL1 protein and gene can be found in FIGS. 2A and 2B.

Reference to a gene encompasses naturally occurring or endogenous versions of the gene, including wild type, polymorphic or allelic variants or mutants (e.g., germline mutation, somatic mutation) of the gene, which can be found in a subject. In an embodiment, the sequence of the biomarker gene is at least about 80%, at least about 85%, at least about 90%, at least about 91%, at least about 92%, at least about 93%, at least about 94%, at least about 95%, at least about 96%, at least about 97%, at least about 98%, or at least about 99% identical to a CXCL1 and/or CXCL5 sequence. Sequence identity can be determined, e.g., by comparing sequences using NCBI BLAST (e.g., Megablast with default parameters).

In an embodiment, the level of expression of the biomarker is determined relative to a control sample, such as the level of expression of the biomarker in normal tissue (e.g., a range determined from the levels of expression of the biomarker observed in normal tissue samples). In an embodiment, the level of expression of the biomarker is determined relative to a control sample, such as the level of expression of the biomarker in samples from other subjects suffering from inflammatory disease. For example, the level of expression of the biomarker in samples from other subjects can be determined to define levels of expression that correlate with sensitivity to treatment with an anti-TNF treatment and/or an anti-IL17 treatment, and the level of expression of the biomarker in the sample from the subject of interest is compared to these levels of expression.

The term “known standard level” or “control level” refers to an accepted or predetermined expression level of the biomarker, for example CXCL1 and/or CXCL5, which is used to compare the expression level of the biomarker in a sample derived from a subject. In one embodiment, the control expression level of the biomarker is the average expression level of the biomarker in samples derived from a population of subjects, e.g., the average expression level of the biomarker in a population of subjects with an inflammatory disease, such as RA. In another embodiment, the population comprises a group of subjects who have not responded to a combination therapy with an anti-TNF treatment and an anti-IL17 treatment, or a group of subjects who express the respective biomarker at high or normal levels. In another embodiment, the control level constitutes a range of expression of the biomarker in normal tissue. In another embodiment, the control level constitutes a range of expression of the biomarker in cells or plasma from a variety of subjects having RA. In another embodiment, “control level” refers also to a pre-treatment level in a subject.

As further information becomes available as a result of routine performance of the methods described herein, population-average values for “control” level of expression of the biomarkers of the present invention may be used. In other embodiments, the “control” level of expression of the biomarkers may be determined by determining the expression level of the respective biomarker in a subject sample obtained from a subject before the suspected onset of inflammatory disease in the subject, from archived subject samples, and the like.

Control levels of expression of biomarkers of the invention may be available from publicly available databases. In addition, Universal Reference Total RNA (Clontech Laboratories) and Universal Human Reference RNA (Stratagene) and the like can be used as controls. For example, qPCR can be used to determine the level of expression of a biomarker, and an increase in the number of cycles needed to detect expression of a biomarker in a sample from a subject, relative to the number of cycles needed for detection using such a control, is indicative of a low level of expression of the biomarker.

As used herein, the term “subject” or “patient” refers to human and non-human animals, e.g., veterinary patients. The term “non-human animal” includes vertebrates, e.g., mammals, such as non-human primates, mice, rodents, rabbits, sheep, dogs, cats, horses, cows, ovine, canine, feline, equine or bovine species. In an embodiment, the subject is a human (e.g., a human with an inflammatory disease, e.g., RA).

The term “sample” refers to cells, tissues or fluids obtained or isolated from a subject, as well as cells, tissues or fluids present within a subject. The term “sample” includes any body fluid, tissue or a cell or collection of cells from a subject, as well as any component thereof, such as a fraction or an extract. In one embodiment, the tissue or cell is removed from the subject. In another embodiment, the tissue or cell is present within the subject. In an embodiment, the fluid comprises amniotic fluid, aqueous humor, vitreous humor, bile, blood, breast milk, cerebrospinal fluid, cerumen, chyle, cystic fluid, endolymph, feces, gastric acid, gastric juice, lymph, mucus, nipple aspirates, pericardial fluid, perilymph, peritoneal fluid, plasma, pleural fluid, pus, saliva, sebum, semen, sweat, serum, sputum, synovial fluid, tears, urine, vaginal secretions, or fluid collected from a biopsy. In one embodiment, the sample contains protein (e.g., proteins or peptides) from the subject. In another embodiment, the sample contains RNA (e.g., mRNA) from the subject or DNA (e.g., genomic DNA molecules) from the subject.

Various aspects of the invention are described in further detail in the following subsections.

I. Prediction of Responsiveness to a Combination Therapy Comprising an Anti-TNF Treatment and an Anti-IL17 Treatment in Subjects with Inflammatory Disease, and Related Methods.

In various aspects, the invention provides a method for determining whether a subject having an inflammatory disease will respond to treatment with a combination therapy comprising an anti-TNF treatment and an anti-IL17 treatment. The method includes the steps of determining a level of expression of at least one of a CXCL1 and a CXCL5 marker in a sample obtained from the subject; and comparing the level of expression of the marker(s) to the level of expression of a control marker. A higher level of expression of at least one of the CXCL1 and the CXCL5 markers, as compared to the level of expression of the control marker, indicates that the combination therapy will be effective in treating the subject. Alternatively, a lower level of expression of at least one of the CXCL1 and the CXCL5 markers after a combination therapy comprising an anti-TNF treatment and an anti-IL17 treatment, as compared to the level of expression of the control marker before treatment with the combination therapy, indicates that the combination therapy will be effective in treating the subject.

In another aspect, the present invention provides a method of determining whether a subject having an inflammatory disease will respond to treatment with a combination therapy comprising an anti-TNF treatment and an anti-IL17 treatment. The method includes the steps of processing a sample obtained from the subject such that the sample is transformed, thereby allowing the determination of a level of expression of at least one of a CXCL1 and a CXCL5 marker and comparing the level of expression of the marker(s) to the level of expression of a control marker, e.g., a normal or disease standard or range of laboratory values). A higher level of expression of at least one of the CXCL1 and the CXCL5 markers, as compared to the level of expression of the control marker, indicates that the combination therapy will be effective in treating the subject. Alternatively, a lower level of expression of at least one of the CXCL1 and the CXCL5 markers after a combination therapy comprising an anti-TNF treatment and an anti-IL17 treatment, as compared to the level of expression of the control marker, indicates that the combination therapy will be effective in treating the subject.

In still another aspect, the present invention provides a method of treating a subject having an inflammatory disease with a combination therapy comprising an anti-TNF treatment and an anti-IL17 treatment. The method includes the steps of selecting a subject exhibiting a higher level of expression of at least one of a CXCL1 and a CXCL5 marker as compared to a level of expression of a control marker and administering a therapeutically effective amount of the combination therapy to the subject. Alternatively, a lower level of expression of at least one of the CXCL1 and the CXCL5 markers after a combination therapy comprising an anti-TNF treatment and an anti-IL17 treatment, as compared to the level of expression of the control marker, indicates that the combination therapy will be effective in treating the subject.

In still another aspect, the present invention provides a method of contraindicating a subject having an inflammatory disease from a combination therapy comprising an anti-TNF treatment and an anti-IL17 treatment. The method includes the steps of selecting a subject exhibiting a lower level of expression of at least one of a CXCL1 and a CXCL5 marker as compared to a level of expression of a control marker, or a normal range of laboratory values.

In yet another aspect, the present invention provides a method for monitoring the effectiveness of a treatment with a combination therapy comprising an anti-TNF treatment and an anti-IL17 treatment. The method includes the steps of determining a level of expression of at least one of a CXCL1 and a CXCL5 marker in a sample obtained from a subject following administering a therapeutically effective amount of the combination therapy to the subject and comparing the level of expression of the marker(s) to a level of expression of a control marker, e.g., a normal range of laboratory values. A lower level of expression of at least one of the CXCL1 and the CXCL5 markers, as compared to the level of expression of the control marker, indicates that the combination therapy has been effective in treating the subject.

In another aspect, the present invention provides a method of selecting a subject for participation in a clinical trial for a combination therapy comprising an anti-TNF treatment and an anti-IL17 treatment for the treatment of an inflammatory disease. The method includes the steps of determining a level of expression of at least one of a CXCL1 and a CXCL5 marker in a sample obtained from the subject and comparing the level of expression of the marker(s) to a level of expression of a control marker. A higher level of expression of at least one of the CXCL1 and the CXCL5 markers, as compared to the level of expression of the control marker, indicates that the subject is suitable for participation in the clinical trial. Alternatively, a lower level of expression of at least one of the CXCL1 and the CXCL5 markers after a combination therapy comprising an anti-TNF treatment and an anti-IL17 treatment, as compared to the level of expression of the control marker, indicates that the combination therapy will be effective in treating the subject in the clinical trial.

In still another aspect, the present invention provides a method for identifying a combination therapy comprising an anti-TNF treatment and an anti-IL17 treatment suitable for treating a subject having an inflammatory disease. The method includes the steps of determining a level of expression of at least one of the CXCL1 and the CXCL5 markers in a sample obtained from the subject and comparing the level of expression of the marker(s) to a level of expression of a control marker. A higher level of expression of at least one of the CXCL1 and the CXCL5 markers, as compared to the level of expression of the control marker, indicates that the combination therapy will be effective in treating the subject. The method can include testing a plurality of different combination therapies. Alternatively, a lower level of expression of at least one of the CXCL1 and the CXCL5 markers after the combination therapy is administered to the subject, as compared to the level of expression of the control marker pre-treatment with the combination therapy, indicates that the combination therapy will be effective in treating the subject.

In yet another aspect, the present invention provides a method of determining whether a subject having an inflammatory disease will respond to treatment with a combination therapy comprising an anti-TNFα antibody and an anti-IL17 antibody. The method includes the steps of determining a level of expression of at least one of a CXCL1 and a CXCL5 marker in a sample obtained from the subject using a reagent that interacts with at least one of the CXCL1 and the CXCL5 markers and transforms the sample such that at least one of the CXCL1 and the CXCL5 markers can be detected and comparing the level of expression of at least one of the CXCL1 and the CXCL5 markers to the level of expression of a control marker. A higher level of expression of at least one of the CXCL1 and the CXCL5 markers, as compared to the level of expression of the control marker, e.g., a normal range of laboratory values, indicates that the combination therapy will be effective in treating the subject. Alternatively, a lower level of expression of at least one of the CXCL1 and the CXCL5 markers after a combination therapy comprising an anti-TNF treatment and an anti-IL17 treatment has been administered, as compared to the level of expression of the control marker, indicates that the combination therapy will be effective in treating the subject.

In still yet another aspect, the present invention provides a kit for (i) determining whether a subject having an inflammatory disease will respond to treatment with a combination therapy comprising an anti-TNF treatment and an anti-IL17 treatment; (ii) monitoring the effectiveness of the combination therapy; (iii) selecting a subject for participation in a clinical trial for the combination therapy; and/or (iv) identifying a combination therapy comprising an anti-TNF treatment and an anti-IL17 treatment for a subject having an inflammatory disease. The kit includes reagents for determining a level of expression of at least one of a CXCL1 and a CXCL5 marker in a sample obtained from the subject and a control marker, e.g., a normal range of values. The kit also includes instructions for (i) determining whether the subject will respond to the combination therapy; (ii) monitoring the effectiveness of the combination therapy; (iii) selecting a subject for participation in a clinical trial for the combination therapy; and/or (iv) identifying a combination therapy comprising an anti-TNF treatment and an anti-IL17 treatment for a subject having an inflammatory disease. Instructions can correspond to any one or more of the aspects described herein.

Any suitable analytical method, can be utilized in the methods of the invention to assess (directly or indirectly) the level of expression of a biomarker in a sample. In an embodiment, a difference is observed between the level of expression of a biomarker, as compared to the control level of expression of the biomarker. In one embodiment, the difference is greater than the limit of detection of the method for determining the expression level of the biomarker. In further embodiments, the difference is greater than or equal to the standard error of the assessment method, e.g., the difference is at least about 2-, about 3-, about 4-, about 5-, about 6-, about 7-, about 8-, about 9-, about 10-, about 15-, about 20-, about 25-, about 100-, about 500- or about 1000-fold greater than the standard error of the assessment method. In an embodiment, the level of expression of the biomarker in a sample as compared to a control level of expression is assessed using parametric or nonparametric descriptive statistics, comparisons, regression analyses, and the like.

In an embodiment, a difference in the level of expression of the biomarker in the sample derived from the subject is detected relative to the control, and the difference is about 5%, about 10%, about 15%, about 20%, about 25%, about 30%, about 40%, about 50%, about 60%, about 70%, about 80%, about 90%, about 100%, about 150%, about 200%, about 250%, about 300%, about 350%, about 400%, about 450%, about 500%, about 600%, about 700%, about 800%, about 900% or about 1000% greater than the expression level of the biomarker in the control sample.

In an embodiment, a difference in the level of expression of the biomarker in the sample derived from the subject is detected relative to the control, and the difference is about 5%, about 10%, about 15%, about 20%, about 25%, about 30%, about 40%, about 50%, about 60%, about 70%, about 80%, or about 90% less than the expression level of the biomarker in the control sample.

The level of expression of a biomarker, for example CXCL1 and/or CXCL5, in a sample obtained from a subject may be assayed by any of a wide variety of techniques and methods, which transform the biomarker within the sample into a moiety that can be detected and/or quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, immunoblotting, Western blotting, Northern blotting, electron microscopy, mass spectrometry, e.g., MALDI-TOF and SELDI-TOF, immunoprecipitations, immunofluorescence, immunohistochemistry, enzyme linked immunosorbent assays (ELISAs), e.g., amplified ELISA, quantitative blood based assays, e.g., serum ELISA, quantitative urine based assays, flow cytometry, Southern hybridizations, array analysis, and the like, and combinations or sub-combinations thereof.

In one embodiment, the level of expression of the biomarker in a sample is determined by detecting a transcribed polynucleotide, or portion thereof, e.g., mRNA, or cDNA, of the biomarker gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, quantitative PCR analysis, RNase protection assays (Melton et al., Nuc. Acids Res. 12:7035), Northern blotting and in situ hybridization. Other suitable systems for mRNA sample analysis include microarray analysis (e.g., using Affymetrix's microarray system or Illumina's BeadArray Technology).

In one embodiment, the level of expression of the biomarker is determined using a nucleic acid probe. The term “probe”, as used herein, refers to any molecule that is capable of selectively binding to a specific biomarker. Probes can be synthesized by one of skill in the art, or derived from appropriate biological preparations. Probes can be specifically designed to be labeled, by addition or incorporation of a label. Examples of molecules that can be utilized as probes include, but are not limited to, RNA, DNA, proteins, antibodies, and organic molecules.

As indicated above, isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to the biomarker mRNA. The nucleic acid probe can be, for example, a full-length cDNA, or a portion thereof, such as an oligonucleotide of at least about 7, 10, 15, 20, 25, 30, 35, 40, 45, 50, 100, 250 or about 500 nucleotides in length and sufficient to specifically hybridize under appropriate hybridization conditions to the biomarker genomic DNA. In a particular embodiment, the probe will bind the biomarker genomic DNA under stringent conditions. Such stringent conditions, for example, hybridization in 6× sodium chloride/sodium citrate (SSC) at about 45° C., followed by one or more washes in 0.2×SSC, 0.1% SDS at 50-65° C., are known to those skilled in the art and can be found in Current Protocols in Molecular Biology, Ausubel et al., eds., John Wiley & Sons, Inc. (1995), sections 2, 4, and 6, the teachings of which are hereby incorporated by reference herein. Additional stringent conditions can be found in Molecular Cloning: A Laboratory Manual, Sambrook et al., Cold Spring Harbor Press, Cold Spring Harbor, N.Y. (1989), chapters 7, 9, and 11, the teachings of which are hereby incorporated by reference herein.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface, for example, in an Affymetrix gene chip array, and the probe(s) are contacted with mRNA. A skilled artisan can readily adapt mRNA detection methods for use in determining the level of the biomarker mRNA.

The level of expression of the biomarker in a sample can also be determined using methods that involve the use of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules. These approaches are especially useful for the detection of nucleic acid molecules if such molecules are present in very low numbers. In particular aspects of the invention, the level of expression of the biomarker is determined by quantitative fluorogenic RT-PCR (e.g., the TaqMan™ System). Such methods typically utilize pairs of oligonucleotide primers that are specific for the biomarker. Methods for designing oligonucleotide primers specific for a known sequence are well known in the art.

The expression levels of biomarker mRNA can be monitored using a membrane blot (such as used in hybridization analysis such as Northern, Southern, dot, and the like), or microwells, sample tubes, gels, beads or fibers (or any solid support comprising bound nucleic acids). See, for example, U.S. Pat. Nos. 5,770,722; 5,874,219; 5,744,305; 5,677,195; and 5,445,934, the entire contents of which as they relate to these assays are incorporated herein by reference. The determination of biomarker expression level may also comprise using nucleic acid probes in solution.

In one embodiment of the invention, microarrays are used to detect or quantify the level of expression of a biomarker. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, e.g., U.S. Pat. Nos. 6,040,138; 5,800,992; 6,020,135; 6,033,860; and 6,344,316, the entire contents of which as they relate to these assays are incorporated herein by reference. High-density oligonucleotide arrays are particularly useful for determining the gene expression profile for a large number of RNA's in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like.

Other known methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, and Western blotting.

Proteins from samples can be isolated using a variety of techniques, including those well known to those of skill in the art. The protein isolation methods employed can, for example, be those described in Harlow and Lane (Harlow and Lane, 1988, Antibodies: A Laboratory Manual, Cold Spring Harbor Laboratory Press, Cold Spring Harbor, N.Y.).

In one embodiment, antibodies, or antibody fragments, are used in methods such as Western blots or immunofluorescence techniques to detect the expressed proteins. Antibodies for determining the expression of the biomarkers of the invention are commercially available.

Anti-CXCL1 antibodies are readily available from a number of commercial suppliers. For example, EMD Millipore: AP1151-100UG, Everest Biotech: EB09637, Lifespan Biosciences: LS-B2843, LS-B2513, LS-C108147, eBioscience: 50-7519-42, 50-7519-41, AbD Serotec: AAM40B, AAM40, AAR22B, Thermo Fisher Scientific, Inc.: PA1-32959, PA1-32924, PA1-20861, Abbiotec: 251349, 12335-1-AP, AP08852PU-N, NovaTeinBio: 63059, Abgent: AT1688a, Aviva Systems Biology: AVARP07032_P050, OASA08635, OAEB00281, United States Biological: C8297-97A, C8298-01B, C8298-01C, Creative Biomart: CAB-1005 MH, CAB-3086 MH, CAB-115 MH, Novus Biologicals: NBP1-61297, NBP1-51486, NBP1-19301, Abnova: H00002919-M01, H00002919-D01P, H00002919-M03, Fitzgerald: 70R-10502, ProSci: 31-057, 42-129, 42-196.

Anti-CXCL5 antibodies are readily available from a number of commercial suppliers. For example, Lifespan Biosciences: LS-B5529, AbD Serotec: AHP1279, AAM42, AHP1279B, Proteintech Group: 10809-1-AP, PA1-29657, Biorbyt: orb13909, orb13450, Acris Antibodies: AM31037PU-N, PP1003B2, PP1003P1, NovaTeinBio: 63066, AT1694a, AT1693a, Aiva Systems Biology: OASA07658, OASA08449, OASA07657, United States Biological: C8297-98H1, C8297-98H, E2275-07, Creative Biomart: CAB-5426 MH, CAB-5425 MH, Novus Biologicals: 33140002, Abnova: H00006374-M05, H00006374-M03, H00006374-B01.

For example, in one embodiment, the methods of the invention may comprise contacting a sample from the subject with an antibody that specifically binds to CXCL1 and/or CXCL5, forming a complex between the antibody and CXCL1 and/or CLXCL5, adding a detection reagent or antibody that is labeled and reactive with the antibody that binds to CXCL1 and/or CXCL5 to detect the complex, washing to remove any unbound detection reagent or antibody, converting the label to the detectable signal and comparing the level of CXCL1 and/or CXCL5 measured to a reference level of CXCL1 and/or CXCL5 obtained from a control sample.

In one embodiment, the antibody or protein can be immobilized on a solid support for Western blots and immunofluorescence techniques. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

Other standard methods include immunoassay techniques which are well known to one of ordinary skill in the art and may be found in Principles And Practice Of Immunoassay, 2nd Edition, Price and Newman, eds., MacMillan (1997) and Antibodies, A Laboratory Manual, Harlow and Lane, eds., Cold Spring Harbor Laboratory, Ch. 9 (1988).

In one embodiment of the invention, proteomic methods, e.g., mass spectrometry, are used. Mass spectrometry is an analytical technique that consists of ionizing chemical compounds to generate charged molecules (or fragments thereof) and measuring their mass-to-charge ratios. In a typical mass spectrometry procedure, a sample is obtained from a subject, loaded onto the mass spectrometry, and its components (e.g., the biomarker) are ionized by different methods (e.g., by impacting them with an electron beam), resulting in the formation of charged particles (ions). The mass-to-charge ratio of the particles is then calculated from the motion of the ions as they transit through electromagnetic fields.

For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis biomarkers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to determine the expression level of a biomarker at the protein level.

Furthermore, in vivo techniques for determination of the expression level of the biomarker include introducing into a subject a labeled antibody directed against the biomarker, which binds to and transforms the biomarker into a detectable molecule. As discussed above, the presence, level, or even location of the detectable biomarker in a subject may be detected by standard imaging techniques.

In general, where a difference in the level of expression of a biomarker and the control is to be detected, it is preferable that the difference between the level of expression of the biomarker in a sample from a subject having an inflammatory disease (e.g., RA) and being treated with an anti-TNF treatment and an anti-IL17 treatment, or being considered for such treatment, and the amount of the biomarker in a control sample, is as great as possible. Although this difference can be as small as the limit of detection of the method for determining the level of expression, it is preferred that the difference be greater than the limit of detection of the method or greater than the standard error of the assessment method, and preferably a difference of at least about 2-, about 3-, about 4-, about 5-, about 6-, about 7-, about 8-, about 9-, about 10-, about 15-, about 20-, about 25-, about 100-, about 500-, 1000-fold greater than the standard error of the assessment method. Alternatively, the difference be greater than the limit of detection of the method or greater than the standard error of the assessment method, and preferably a difference of at least about 2-, about 3-, about 4-, about 5-, about 6-, about 7-, about 8-, about 9-, about 10-, about 15-, about 20-, about 25-, about 100-, about 500-, 1000-fold less than the standard error of the assessment method.

Any suitable sample obtained from a subject having an inflammatory disease (e.g., RA) may be used to assess the level of expression, including a lack of expression, of the biomarker, for example CXCL1 and/or CXCL5. For example, the sample may be any fluid or component thereof, such as a fraction or extract, e.g., blood, plasma, lymph, synovial fluid, cystic fluid, urine, nipple aspirates, or fluids collected from a biopsy, amniotic fluid, aqueous humor, vitreous humor, bile, blood, breast milk, cerebrospinal fluid, cerumen, chyle, cystic fluid, endolymph, feces, gastric acid, gastric juice, mucus, pericardial fluid, perilymph, peritoneal fluid, plasma, pleural fluid, pus, saliva, sebum, semen, sweat, serum, sputum, synovial fluid, joint tissue or fluid, tears, or vaginal secretions obtained from the subject. In a typical situation, the fluid may be blood, or a component thereof, obtained from the subject, including whole blood or components thereof, including, plasma, serum, and blood cells, such as red blood cells, white blood cells and platelets. In another typical situation, the fluid may be synovial fluid, joint tissue or fluid, or any other sample reflective of an inflammatory disease (e.g., RA). The sample may also be any tissue or component thereof, connective tissue, lymph tissue or muscle tissue obtained from the subject.

Techniques or methods for obtaining samples from a subject are well known in the art and include, for example, obtaining samples by a mouth swab or a mouth wash; drawing blood; obtaining a biopsy; or obtaining synovial fluid or other sample from a subject suffering from inflammatory disease (e.g., skin, as in the case of psoriasis or psoriatic arthritis). Isolating components of fluid or tissue samples (e.g., cells or RNA or DNA) may be accomplished using a variety of techniques. After the sample is obtained, it may be further processed.

II. Treatment with a Combination Therapy Comprising an Anti-TNF Treatment and an Anti-IL17 Treatment.

Given the observation that the expression levels of CXCL1 and/or CXCL5 in a subject having inflammatory disease (e.g., RA) influences the responsiveness of the subject to a combination therapy of an anti-TNF treatment and an anti-IL17 treatment, a skilled artisan can select an appropriate treatment regimen for the subject based on the expression levels of the CXCL1 and/or CXCL5 biomarkers in the subject.

Accordingly, the present invention provides methods for treating a subject having an inflammatory disease with a combination therapy comprising an anti-TNF treatment and an anti-IL17 treatment.

In an embodiment, the subject may have been previously treated with a monotherapy comprising an anti-TNF treatment or an anti-IL17 treatment, a combination therapy comprising an anti-TNF treatment and an anti-IL17 treatment, and/or an alternative therapy. In other embodiments, the subject may be under consideration for treatment with a combination therapy comprising an anti-TNF treatment and an anti-IL17 treatment for the first time. The level of expression of at least one of a CXCL1 marker and a CXCL5 marker is determined. If the level of expression of at least one of the CXCL1 and CXCL5 biomarker is determined to be higher than a control level of expression, treatment with a combination therapy comprising an anti-TNF treatment and an anti-IL17 treatment is likely to be efficacious. However, it is not necessary that all of the biomarkers assayed have a high level of expression as compared to the respective control. For example, while certain biomarkers may be present at normal or high levels of expression, treatment with a combination therapy comprising an anti-TNF treatment and an anti-IL17 treatment, may be indicated when, for example, either CXCL1 or CXCL5 is expressed at a lower level than a control level.

The treatment regimen for a combination therapy comprising an anti-TNF treatment and an anti-IL17 treatment, that is selected typically includes at least one of the following parameters and more typically includes many or all of the following parameters: the dosage, the formulation, the route of administration and/or the frequency of administration. Selection of the particular parameters of the treatment regimen can be based on known treatment parameters for an anti-TNF therapy and an anti-IL17 therapy previously established in the art such as those described in the Dosage and Administration protocols set forth in the FDA Approved Label for Adalimumab®, infliximab, and secukinumab, the entire contents of which are incorporated herein by reference. Various modifications to dosage, formulation, route of administration and/or frequency of administration can be made based on various factors including, for example, the disease, age, sex, and weight of the patient, as well as the severity or stage of inflammatory disease (e.g., RA) by methods known in the art.

The anti-TNF treatment and the anti-IL17 treatment may be administered at the same time or at different times. A combination therapy can include the simultaneous or near simultaneous administration of an anti-TNF therapy and an anti-IL17 therapy. In other embodiments, a combination therapy can include the administration of an anti-TNF therapy followed by an anti-IL17 therapy, where the separation in such that both the anti-TNF therapy and the anti-IL17 therapy act concomitantly and/or achieve a synergistic effect. In other embodiments, a combination therapy can include the administration of an anti-IL17 therapy followed by an anti-TNF therapy, where the separation in such that both the anti-TNF therapy and the anti-IL17 therapy act concomitantly and/or achieve a synergistic effect. In an embodiment, the combination therapy includes both an anti-TNF therapy and an anti-IL17 therapy in the same formulation (e.g., as a single molecule or as two separate molecules). In other embodiments, the combination therapy includes two separate formulations, one including an anti-TNF therapy and another including an anti-IL17.

In one embodiment, the combination therapy can be a DVD-Ig binding protein (e.g., and anti-TNF-anti-IL17 DVD-Ig) as described in, for example, WO/2010/102251, incorporated herein by reference in its entirety.

In one embodiment, the combination therapy can be a DVD-Ig binding protein (e.g., and anti-TNF-anti-IL17 DVD-Ig) as described in, for example, WO/2010/102251, incorporated herein by reference in its entirety.

As used herein, the term “therapeutically effective amount” means an amount of an anti-TNF treatment and an anti-IL17 treatment as described herein, which is capable of treating inflammatory disease (e.g., RA). The dose of a therapy to be administered according to this invention will, of course, be determined in light of the particular circumstances surrounding the case including, for example, the therapy administered, the route of administration, condition of the patient, and the pathological condition being treated, for example, the severity of the RA in the subject.

For administration to a subject, the combination therapy typically is formulated into a pharmaceutical composition comprising an anti-TNF treatment and an anti-IL17 treatment and a pharmaceutically acceptable carrier. Therapeutic compositions typically should be sterile and adequately stable under the conditions of manufacture and storage.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral (e.g., intravenous, intramuscular, subcutaneous, intrathecal) administration (e.g., by injection or infusion). Depending on the route of administration, the active compound may be coated in a material to protect the compound from the action of acids and other natural conditions that may inactivate the compound.

There are numerous types of anti-inflammatory approaches that can be used in conjunction with the combination therapy comprising an anti-TNF treatment and an anti-IL17 treatment, according to the invention. These include, for example, nonsteroidal anti-inflammatory drugs (NSAIDs), steroids, disease-modifying antirheumatic drugs (DMARDs) including methotrexate (Trexall), leflunomide (Arava), hydroxychloroquine (Plaquenil), sulfasalazine (Azulfidine) and minocycline (Dynacin, Minocin), and immunosuppressants including azathioprine (Imuran, Azasan), cyclosporine (Neoral, Sandimmune, Gengraf) and cyclophosphamide (Cytoxan).

The methods of the invention can employ these approaches to treat the same types of inflammatory disease as those for which they are known in the art to be used, as well as others, as can be determined by those of skill in this art. Also, these approaches can be carried out according to parameters (e.g., regimens and doses) that are similar to those that are known in the art for their use. However, as is understood in the art, it may be desirable to adjust some of these parameters, due to the additional use of an anti-TNF treatment and an anti-IL17 treatment, with these approaches. For example, if another drug is normally administered as a sole therapeutic agent, when combined with an anti-TNF treatment and an anti-IL17 treatment according to the invention, it may be desirable to decrease the dosage of the drug, as can be determined by those of skill in this art.

### III. Kits of the Invention

In still yet another aspect, the present invention provides a kit for (i) determining whether a subject having an inflammatory disease will respond to treatment with a combination therapy comprising an anti-TNF treatment and an anti-IL17 treatment, (ii) monitoring the effectiveness of the combination therapy, (iii) selecting a subject for participation in a clinical trial for the combination therapy for the inflammatory disease, or (iv) identifying a combination therapy comprising an anti-TNF treatment and an anti-IL17 treatment for a subject having an inflammatory disease. The kit includes reagents for determining a level of expression of at least one of a CXCL1 and a CXCL5 marker in a sample obtained from the subject and a control marker. The kit also includes instructions for (i) determining whether the subject will respond to the combination therapy comprising, (ii) monitoring the effectiveness of the combination therapy, (iii) selecting a subject for participation in a clinical trial for the combination therapy, or (iv) identifying a combination therapy comprising an anti-TNF treatment and an anti-IL17 treatment for a subject having an inflammatory disease. Instructions can correspond to any one or more of the methods described herein.

The kits of the invention can optionally comprise additional components useful for performing the methods of the invention.

By way of example, the kits can comprise reagents for obtaining a biological sample from a subject and/or a control sample.

Furthermore, the kit includes reagents for determining a level of expression of at least one of a CXCL1 and a CXCL5 marker. In one example, the reagents for determining the level of expression of the at least one of a CXCL1 and a CXCL5 marker comprise a probe for amplifying and detecting at least one of the CXCL1 and the CXCL5 marker. In another example, the reagents for determining the level of expression of at least one of a CXCL1 and a CXCL5 marker comprise an antibody, or antigen binding fragment thereof.

With respect to the control marker, the kit can include a predetermined control value (e.g., based on a predetermined population of subjects). Alternatively, the kit can include further reagents and instructions for determining the level of expression of a control in the subject (e.g., a potential candidate for therapy or a subject receiving therapy).

In one embodiment, the reagents for determining the expression level of at least one biomarker in a biological sample from the subject comprise a nucleic acid preparation sufficient to detect expression of a nucleic acid, e.g., mRNA, encoding the biomarker. This nucleic acid preparation includes at least one, and may include more than one, nucleic acid probe or primer, the sequence(s) of which is designed such that the nucleic acid preparation can detect the expression of nucleic acid, e.g., mRNA, encoding the biomarker in the sample from the subject. A preferred nucleic acid preparation includes two or more PCR primers that allow for PCR amplification of a segment of the mRNA encoding the biomarker of interest. In other embodiments, the kit includes a nucleic acid preparation for CXCL1 and/or CXCL5.

The means for determining the expression level of CXCL1 and/or CXCL5 can also include, for example, buffers or other reagents for use in an assay for evaluating expression (e.g., at either the nucleic acid or protein level). The assay can be a bioassay, e.g., an ex vivo assay where a patients cells (e.g., monocytes) are removed and tested in culture with the combination therapy.

In another embodiment, the kit can further comprise a combination therapy comprising an anti-TNF treatment and an anti-IL17 treatment, for treating an inflammatory disease as described herein. By way of example, the combination therapy comprises a DVD-Ig molecule directed against TNF and IL17 or, more particularly against TNFα and IL17.

Preferably, the kit is designed for use with a human subject.

The present invention is further illustrated by the following examples which should not be construed as further limiting. The contents of all references, patents and published patent applications cited throughout this application, as well as the Figures are expressly incorporated herein by reference in their entirety.

### EXAMPLES

### Example 1

### Efficacy of Anti-TNF and Anti-IL17, Alone and in Combination, in a Mouse Collagen Induced Arthritis Model

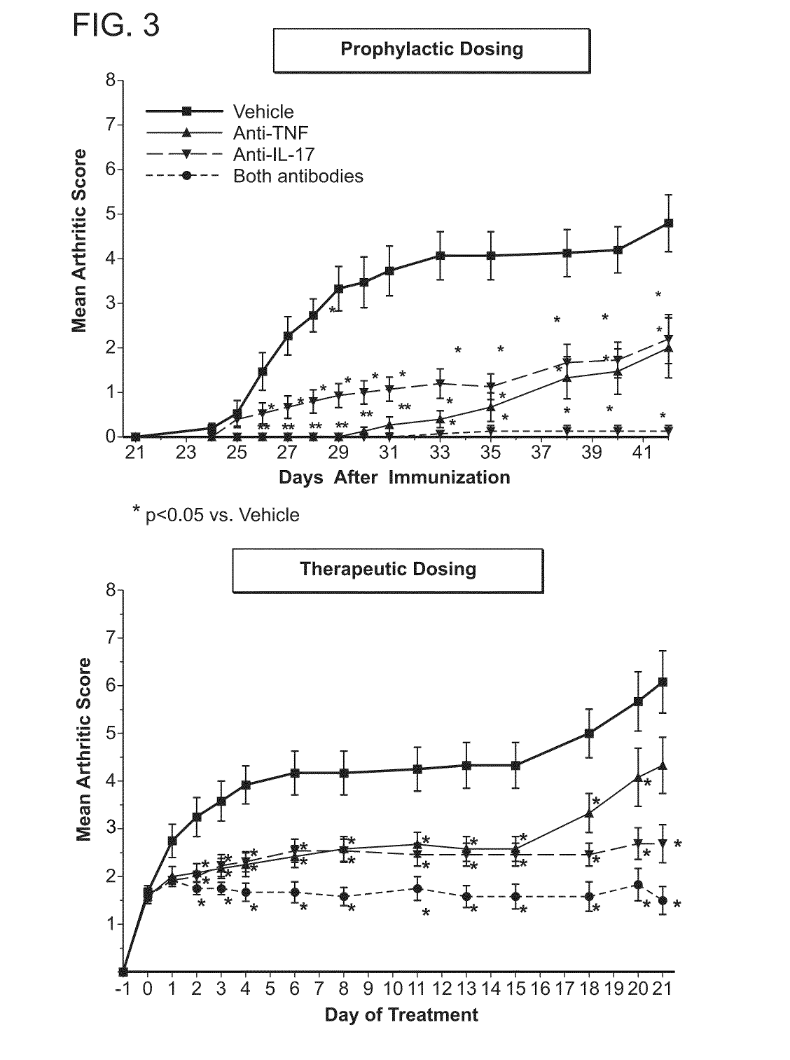

In this example, the efficacy of anti-TNF or anti-IL17, or a combination thereof, was evaluated in a mouse model of collagen-induced arthritis and the results are shown in FIG. 3. Male DBA/1J mice were injected i.d. at the base of the tail with 100 μL of emulsion containing 100 μg of type II bovine collagen dissolved in 0.1N acetic acid and 100 μL of Complete Freund's Adjuvant containing 100 μg of Mycobacterium Tuberculois H37Ra. Mice were boosted 21 days later i.p. with 1.0 mg zymosan A in 200 μL of phosphate buffered saline (PBS). Disease onset occurred within 3 days of the boost. Mice were monitored for arthritis daily for the first week and three times per week thereafter. Each paw was scored by the following criteria: 0=normal; 1=swelling in one site, foot or ankle; 2=swelling in foot and ankle; 3=ankylosis. Scores were summed for all 4 paws of each animal (maximal score of 12) and total score expressed as an average of all animals in each group and expressed as Mean Arthritic Score (MAS). Animals were treated 2×/week with either 12 mg/kg of anti-TNF antibody 8C11, 12 mg/kg of anti-IL17 antibody MAB421, or 12 mg/kg of each antibody administered by intra-peritoneal injection in phosphate buffered saline (PBS). In one experiment, animals received antibody treatments prior to exhibiting arthritic signs in a prophylactic treatment mode. In a second experiment, animals received antibody treatment after onset of arthritic signs in a therapeutic treatment mode. In both types of experiments treatment with anti-TNF or anti-IL17 was partially effective in reducing the arthritis in the paws whereas treatment with the combination of anti-TNF and anti-IL17 conferred greater efficacy than either monotherapy alone.

### Example 2

### Comparison of Bone Protection by Anti-TNF and Anti-IL17, Alone and in Combination, in a Mouse Collagen Induced Arthritis Model

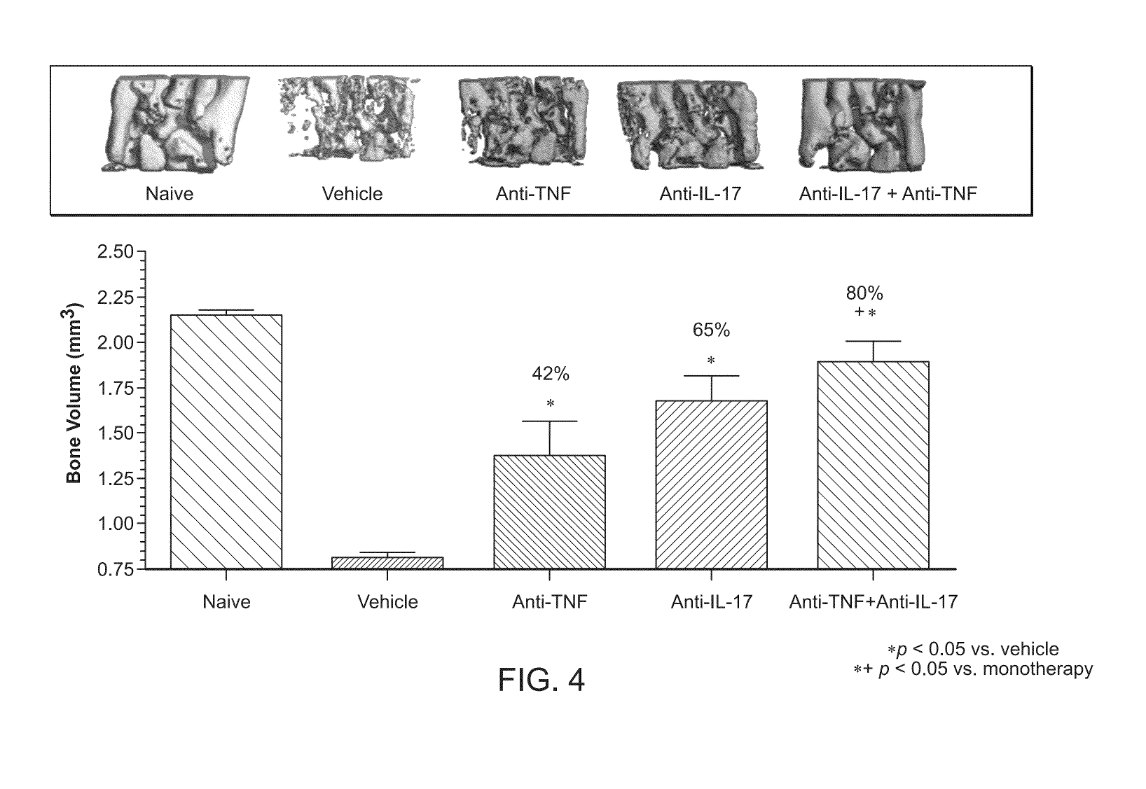

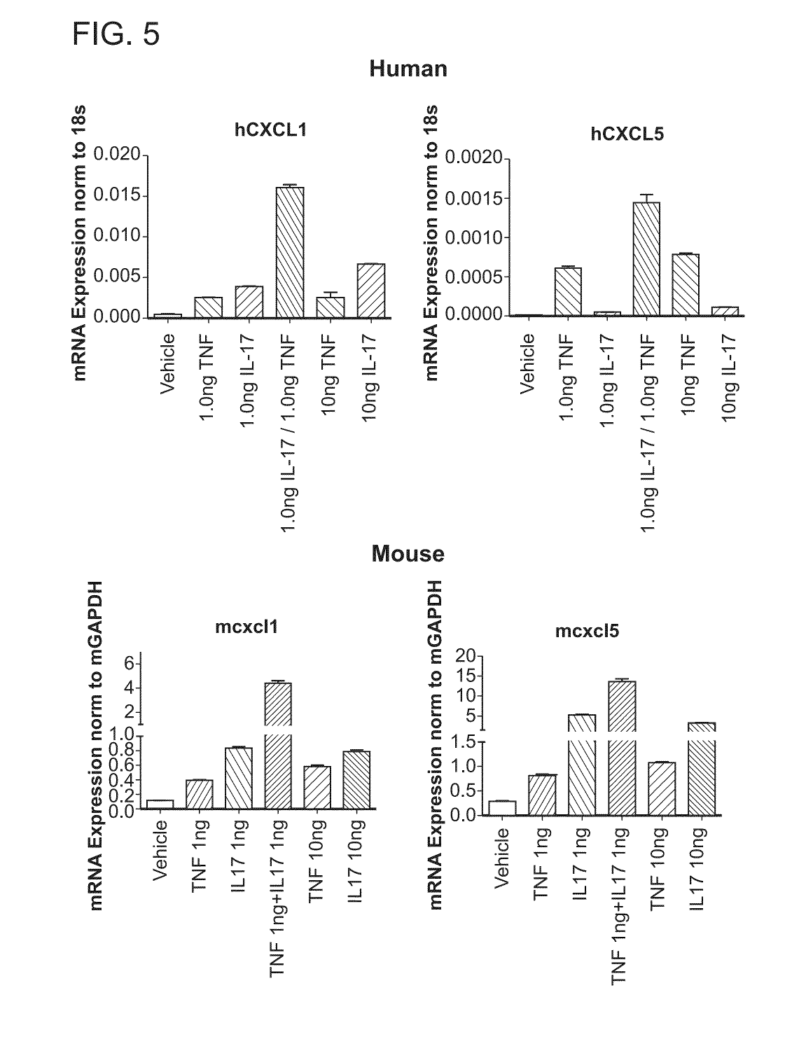

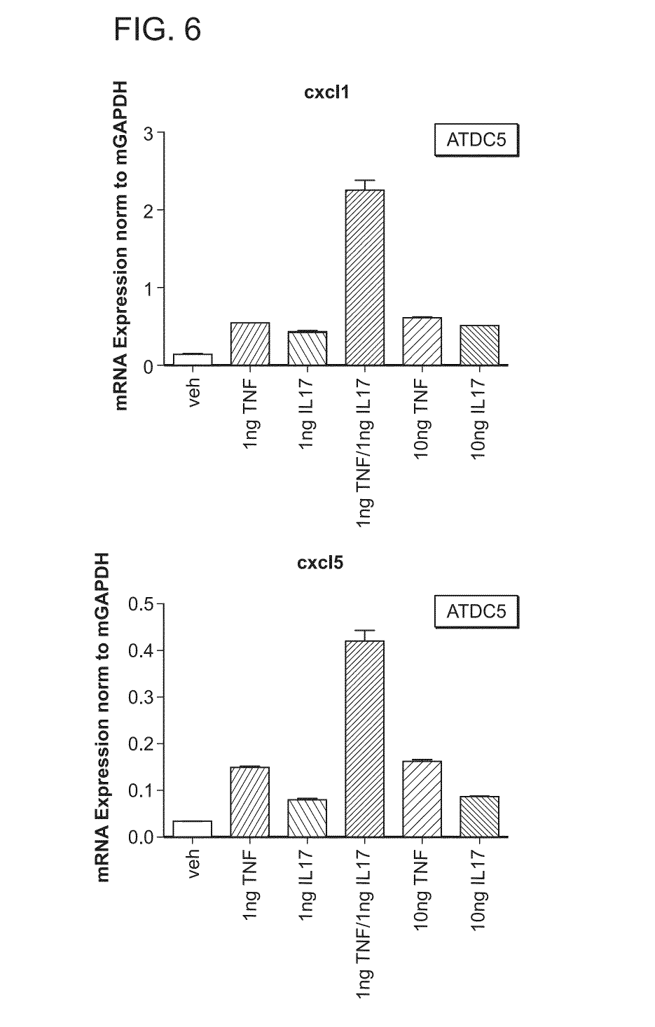

In this example, the greater efficacy of the combined blockade of TNF and IL17 is demonstrated on protection from bone loss and is shown in FIG. 4. Arthritis was induced in the DBA/1J mice as described in Example 1. The level of bone loss was evaluated at the termination of the study, three weeks after onset of arthritic signs. The effect on protection from bone loss was measured in animals receiving therapeutic treatment regimen. Hind paws were removed at the middle of the tibia/fibula and stored in 10% neutral buffered formalin. Paws were imaged using a Scanco μCT40 (Scanco Medical AG) at 55 kVp and 145 μA, utilizing the High Resolution setting (1000 Projections/180° at 2048×2048 Pixel Reconstruction) and Isotropic Voxels with 180 millisecond integration time, resulting in a final isotropic voxel size of 18 μm×18 μm×18 μm. A cylindrical contour was manually drawn around a region of interest from the proximal junction of the calcaneous and navicular bone and extending into the tarsals for a fixed height of 100 slices (1.8 mm). In-house naïve controls have shown this region to give a highly conserved and statistically reproducible volumetric region for analysis. 3-D quantitative evaluation was performed by ScancoAG analytical software for bone volume (mm³) and surface area to volumetric ratio, giving an approximation of tarsal surface roughness (mm⁻¹). Analytical settings of 0.8 sigma gauss and 1.0 were used, with an upper threshold of 1000 and a lower threshold of 320. As is shown in FIG. 4, there was a significant loss in bone volume in arthritic mice receiving vehicle treatment. In contrast, treatment with anti-TNF or anti-IL17 alone resulted in significant protection from bone loss by 42 or 66%, respectively (p value <0.05 vs vehicle control). The combination treatment of anti-TNF and anti-IL17 resulted in a greater protection from bone loss of 80% vs. vehicle (p<0.05).

### Example 3

### TNF and IL17 Interaction in CIA and RA

In this example, gene expression profiling was used as a tool to study biomarkers that reflect the cooperative action of anti-TNF and anti-IL17 therapies. In this case, an 8C11 antibody was used as the anti-TNF therapy and a rat anti-mouse anti-IL17 antibody, MAB421, was used as the anti-IL17 therapy, unless otherwise noted. Using art-known methods, the responses of disease related RNAs were characterized and a cohort that was sensitive to combined anti-TNF and anti-IL17 therapy but substantially less sensitive to anti-TNF or anti-IL17 monotherapy was identified. CXCL1 and CXCL5 were identified as biomarkers because they are stable over time in an easily accessible biological fluid requiring minimal preparation or handling at the clinical site. Using a collagen-induced arthritis (CIA) model in mice, measurements of CXCL1 and CXCL5 biomarkers in whole paw homogenates indicated that a change in RNA level was a good predictor of protein level changes. Furthermore, the results establish that a synergistic effect exists with the combination therapy of an anti-TNF therapy and an anti-IL17 therapy as discussed below.

### Statistical Analysis of CXCL1 and CXCL5—Responsiveness and Unresponsiveness

A CXCL1 and/or CXCL5 gene was considered unresponsive to a given treatment if the p-value (using Student's t-test, without correction for multiple comparisons) for the significance of the difference between treated and diseased fell above 0.05 or the fold change due to treatment was less than 0.1 log (25%), regardless of the significance of the change.

### CIA Model and RNA Preparation

Male DBA/1J mice were injected i.d. at the base of the tail with 100 μL of emulsion containing 100 μg of Type II Bovine Collagen dissolved in 0.1N acetic acid and 100 μL of Complete Freund's Adjuvant containing 100 μg of Mycobacterium Tuberculois H37Ra. Mice were boosted 21 days later i.p. with 1.0 mg Zymosan A in 200 μL of Phosphate buffered saline (PBS). Disease onset occurs within 3 days of the boost.

Mice were monitored for arthritis daily for the first week and three times per week thereafter. Each paw was scored by the following criteria: 0=normal; 1=swelling in one site, foot or ankle; 2=swelling in foot and ankle; 3=ankylosis. Scores are summed for all 4 paws of each animal (maximal score of 12) and total score expressed as an average of all animals in each group and expressed as Mean Arthritic Score. Change in paw swelling was measured with a Caliper Thickness-Gage (Dyer, 310-115). For therapeutic dosing, mice were enrolled into groups at first clinical signs of disease with a maximal score of 2.

RNA was prepared from the skinned rear paws of mice seven days after the first presentation of disease symptoms. Upon enrollment, mice were randomized into treatment cohorts consisting of monotherapy (6 mg/kg, either antibody), combination therapy (6 mg/kg each antibody) or mice treated with an isotype control. Doses were selected based on previous dose-response experiments that determined 6 mg/kg to be the maximum effective dose.

### Pathway Analysis

Comparative pathway analysis used Ingenuity Pathway Assist™ and Fisher's Exact Test method. For the present analysis, the indicated pathways had a significance of <0.05 and at least two regulated transcripts.

### Results:

### IL17 and TNF Appear to Cooperatively Regulate Gene Expression in CIA Mice

Total paw RNA from the CIA mice was analyzed to investigate the interaction of the two cytokine pathways in regulating disease activity. CXCL1 and CXCL5 gene expression was significantly up-regulated in diseased animals compared to healthy animals. Clustering results indicated that mice that received similar treatment regimens had similar RNA expression profiles Animals treated with the anti-IL17 antibody alone clustered with vehicle treated animals indicating a minimal effect on gene expression. The anti-TNF antibody treated mice clustered together, but separately from the mice treated with both anti-TNF and anti-IL17, having an intermediate effect to that of anti-IL17 monotherapy. Thus, based on unbiased clustering analysis there was a gradient of effects across the three treatment regimens.

According to cluster analysis, a minority of disease related genes responded significantly to anti-TNF treatment whereas an even smaller amount responded significantly to anti-IL17 treatment. However, about twice the number of genes responded to the combination therapy as compared to the number of genes that responded to the anti-TNF monotherapy. Further analysis using the same statistical criteria indicated that most of the mRNAs regulated by anti-IL17 alone were also regulated by the combination. By comparison only half the genes regulated by anti-TNF alone were also regulated by the combination.

Pathway Analysis Indicates that Combination Therapy Affects Substantially More Systems than Monotherapy

The increased mRNA regulation observed could have been a consequence of increased potency, leading to detection of more mRNAs within more or less the same pathways affected by the monotherapies. On the other hand, the increased mRNA regulation could have also had qualitative consequences indicating effects on pathways unaffected by the monotherapies. To help distinguish between these two mechanisms, a pathway analysis of the genes regulated in each treatment group was performed using a curated pathway database (Ingenuity Pathway Assist™). This analysis indicated that although several pathways were regulated in common, there were substantially more pathways affected by the combination treatment, indicating additional non-redundant functions attributable to the combination anti-TNF and anti-IL17 treatment.

To determine whether the increases in mRNA levels were reflected in changes in protein levels, the levels of CXCL1 protein and CXCL5 protein were analyzed using Ingenuity Pathway Assist™. There was a substantial disease related increase in both CXCL1 protein and CXCL5 protein levels detected in total paw homogenates. Decreases in these proteins levels occurred in response to anti-TNF and anti-IL17 treatment, alone and in combination, to varying degrees. In all cases there was a greater decrease in CXCL1 protein and CXCL5 protein levels in response to combination therapy compared to either monotherapy. Thus, changes in CXCL1 RNA and CXCL5 RNA levels are qualitatively reliable predictors of CXCL1 protein and CXCL5 protein expression in the mouse CIA model as well as indicators of responsiveness to anti-TNF and anti-IL17 treatments.

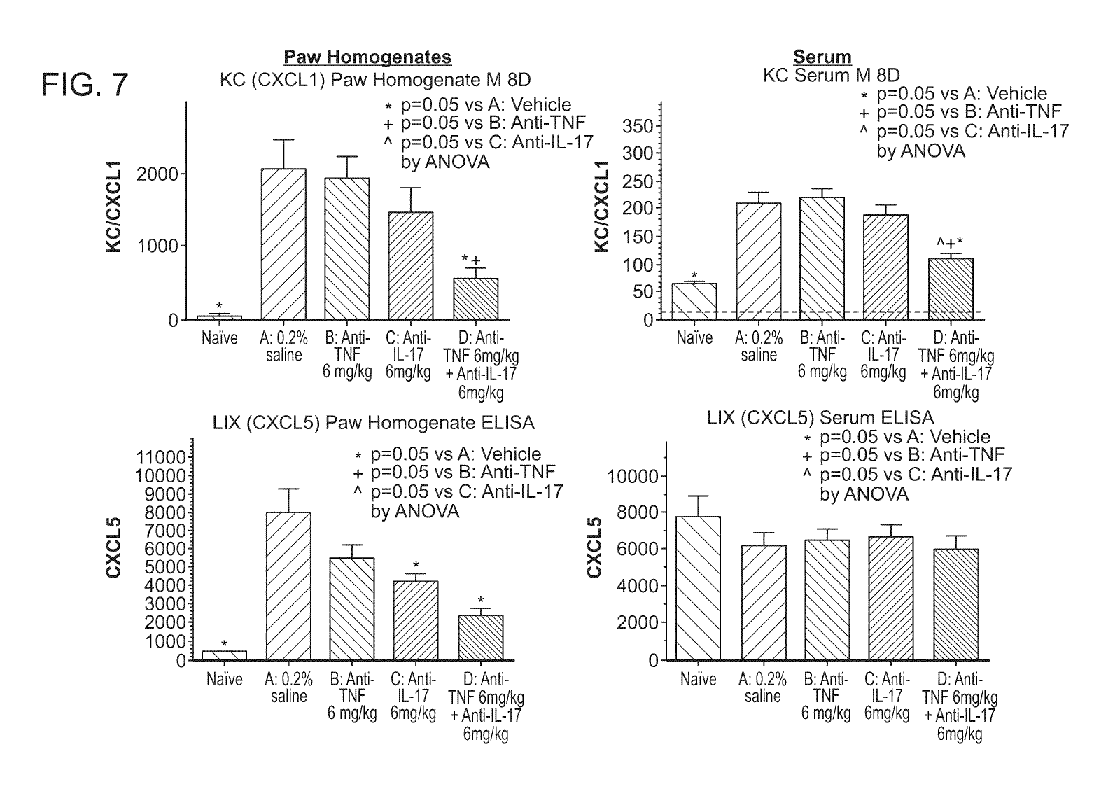

FIG. 7 shows protein expression in mouse paw lysates (left column) and serum (right column). The trends observed in serum generally reflected those observed in the paw lysates for CXCL1. CXCL1 protein levels were up-regulated in the diseased mice, only marginally down-regulated, if at all, by either mono-therapy, but substantially down-regulated by combination treatment. Thus, there was a general correlation between paw and serum chemokine levels.

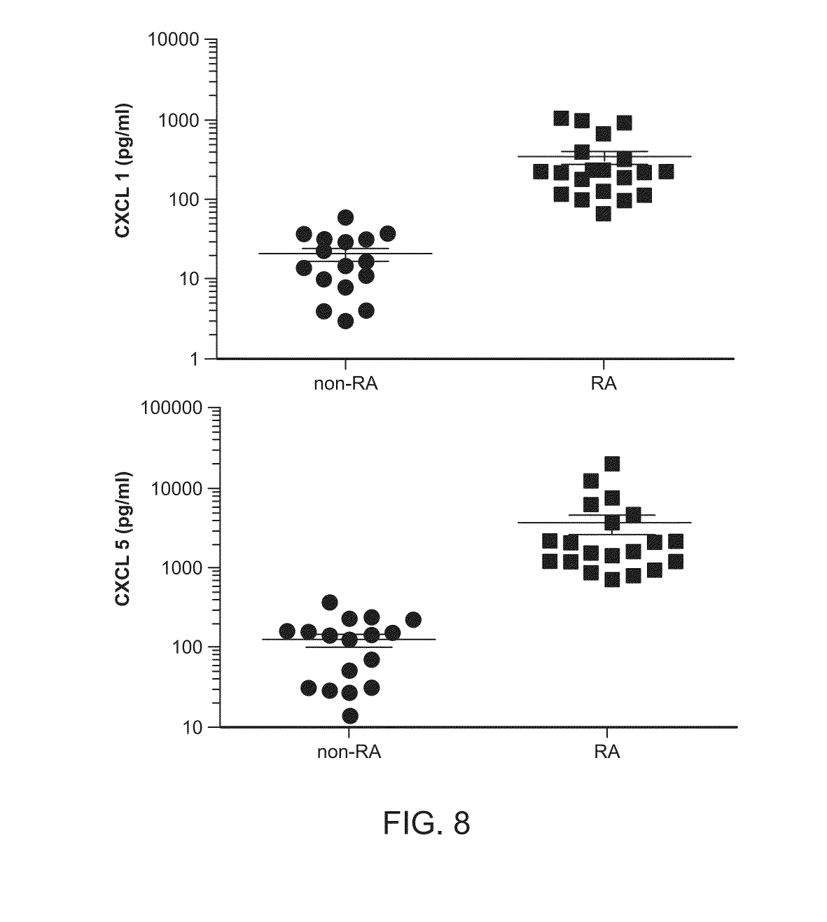

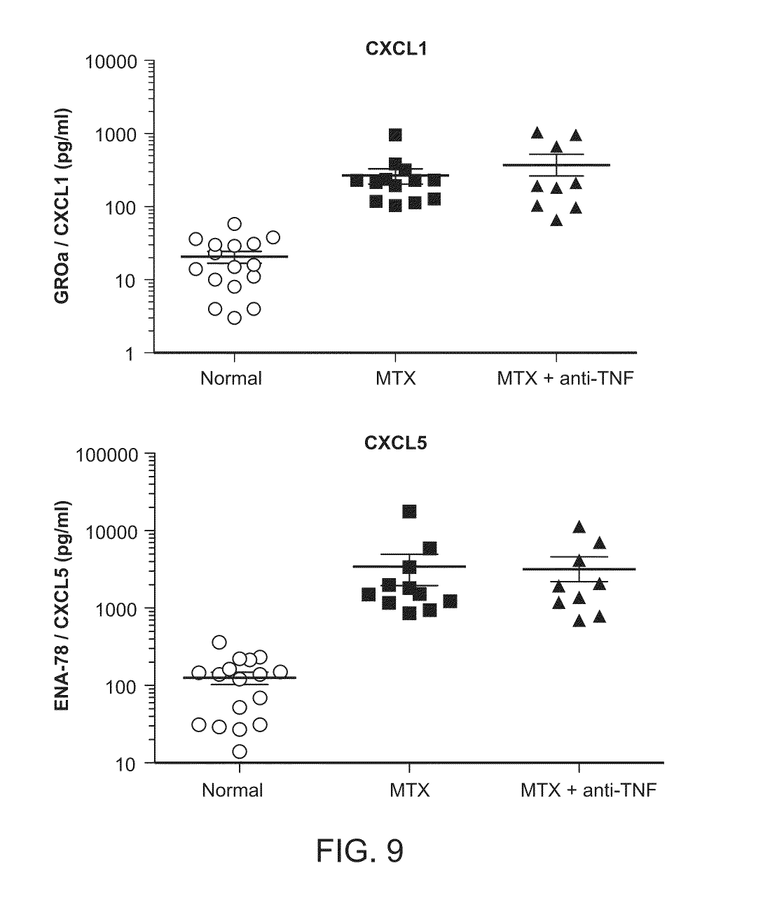

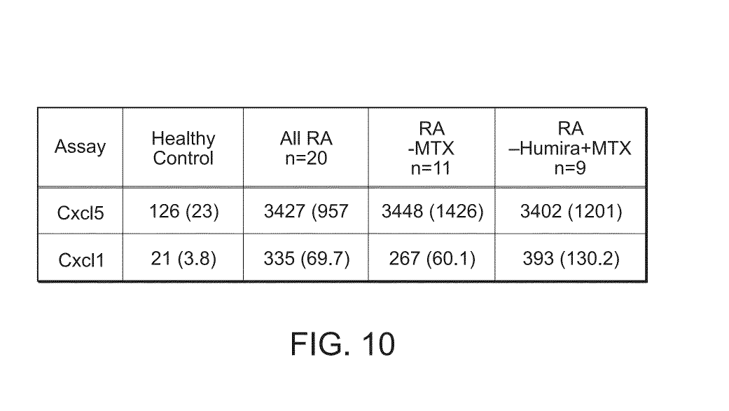

To determine whether secreted CXCL1 and CXCL5 protein levels were similarly regulated in humans, serum samples were obtained from patients with established RA and healthy controls. FIG. 8 shows that CXCL1 and CXCL5 protein levels were highly upregulated in RA patients compared to non-RA controls. Because there is a need for biomarkers that are responsive to existing commercial combination therapies, the RA cohort of the study depicted in FIG. 8 included patients that were being treated with methotrexate alone or Humira® plus methotrexate in order to determine whether the CXCL1 and CXCL5 markers continued to be over-expressed in the presence of an additional TNF blockade. FIG. 9 shows that there is no significant influence of methotrexate alone or the addition of methotrexate to the anti-TNF treatment on CXCL1 and CXCL5 levels. FIG. 10 shows the numerical results of the experiment illustrated in FIG. 9.

### INCORPORATION BY REFERENCE

The contents of all cited references (including literature references, patents, patent applications, databases and websites) that maybe cited throughout this application are hereby expressly incorporated by reference in their entirety for any purpose, as are the references cited therein. The practice of the present invention will employ, unless otherwise indicated, conventional techniques of immunology, molecular biology and cell biology, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

What is claimed is:

1. A method of determining whether a subject having an inflammatory disease will respond to treatment with a combination therapy comprising an anti-TNF treatment and an anti-IL17 treatment, the method comprising the steps of:
determining a level of expression of at least one of a CXCL1 and a CXCL5 marker in a sample obtained from the subject; and
comparing the level of expression of the marker(s) to the level of expression of a control marker,
wherein a higher level of expression of at least one of the CXCL1 and the CXCL5 markers, as compared to the level of expression of the control marker, and/or a lower level of expression of at least one of the CXCL1 and the CXCL5 markers after the combination therapy comprising the anti-TNF treatment and the anti-IL17 treatment has been administered, as compared to the level of expression of the control marker, indicates that the combination therapy will be effective in treating the subject.

2. A method of determining whether a subject having an inflammatory disease will respond to treatment with a combination therapy comprising an anti-TNF treatment and an anti-IL17 treatment, the method comprising the steps of:
processing a sample obtained from the subject such that the sample is transformed, thereby allowing the determination of a level of expression of at least one of a CXCL1 and a CXCL5 marker; and
comparing the level of expression of the marker(s) to the level of expression of a control marker,
wherein a higher level of expression of at least one of the CXCL1 and the CXCL5 markers, as compared to the level of expression of the control marker, indicates that the combination therapy will be effective in treating the subject and/or a lower level of expression of at least one of the CXCL1 and the CXCL5 markers after the combination therapy comprising the anti-TNF treatment and the anti-IL17 treatment has been administered, as compared to the level of expression of the control marker, indicates that the combination therapy will be effective in treating the subject.

3. A method of treating a subject having an inflammatory disease with a combination therapy comprising an anti-TNF treatment and an anti-IL17 treatment, the method comprising the steps of:
selecting a subject exhibiting a higher level of expression of at least one of a CXCL1 and a CXCL5 marker as compared to a level of expression of a control marker and/or selecting a subject exhibiting a lower level of expression of at least one of a CXCL1 and a CXCL5 marker as compared to a level of expression of a control marker in response to the combination therapy; and
administering a therapeutically effective amount of the combination therapy to the subject.

4. A method for monitoring the effectiveness of a treatment with a combination therapy comprising an anti-TNF treatment and an anti-IL17 treatment, the method comprising the steps of:
determining a level of expression of at least one of a CXCL1 and a CXCL5 marker in a sample obtained from a subject following administering a therapeutically effective amount of the combination therapy to the subject, and
comparing the level of expression of the marker(s) to a level of expression of a control marker,
wherein a lower level of expression of at least one of the CXCL1 and the CXCL5 markers, as compared to the level of expression of the control marker, indicates that the combination therapy has been effective in treating the subject.

5. A method of selecting a subject for participation in a clinical trial for a combination therapy comprising an anti-TNF treatment and an anti-IL17 treatment for the treatment of an inflammatory disease, the method comprising the steps of:
determining a level of expression of at least one of a CXCL1 and a CXCL5 marker in a sample obtained from the subject; and
comparing the level of expression of the marker(s) to a level of expression of a control marker,
wherein a higher level of expression of at least one of the CXCL1 and the CXCL5 markers, as compared to the level of expression of the control marker, indicates that the subject is suitable for participation in the clinical trial and/or a lower level of expression of at least one of the CXCL1 and the CXCL5 markers after the combination therapy comprising the anti-TNF treatment and the anti-IL17 treatment has been administered, as compared to the level of expression of the control marker, indicates that the combination therapy will be effective in treating the subject.

6. A method for identifying a combination therapy comprising an anti-TNF treatment and an anti-IL17 treatment suitable for treating a subject having an inflammatory disease, the method comprising the steps of:
determining a level of expression of at least one of the CXCL1 and the CXCL5 marker(s) in a sample obtained from the subject; and
comparing the level of expression of the marker(s) to a level of expression of a control marker,
wherein a higher level of expression of at least one of the CXCL1 and the CXCL5 markers, as compared to the level of expression of the control marker, and/or a lower level of expression of at least one of the CXCL1 and the CXCL5 markers after the combination therapy comprising the anti-TNF treatment and the anti-IL17 treatment has been administered, as compared to the level of expression of the control marker, indicates that the combination therapy will be effective in treating the subject.

7. The method of claim 6, wherein a plurality of different combination therapies are tested.

8. The method of claim 1, wherein the level of expression of at least one of the CXCL1 and the CXCL5 marker(s) in the sample is determined after a predetermined amount of the anti-TNF treatment and the anti-IL17 treatment are administered to the subject.

9. The method of claim 8, wherein the predetermined amount comprises a sub-therapeutic dose of at least one of the anti-TNF treatment and the anti-IL17 treatment.

10. The method of claim 8, wherein the predetermined amount comprises a sub-therapeutic dose of the anti-TNF treatment and the anti-IL17 treatment.

11. The method of claim 8, wherein the predetermined amount comprises a therapeutic dose of at least one the anti-TNF treatment and the anti-IL17 treatment.

12. The method of claim 8, wherein the predetermined amount comprises a therapeutic dose of the anti-TNF treatment and the anti-IL17 treatment.

13. The method of claim 1, wherein the level of expression of the control marker is the level of expression of the control marker in the sample before a predetermined amount of the anti-TNF treatment and/or the anti-IL17 treatment are administered to the subject.

14. The method of claim 1, wherein the level of expression of the control marker is an average level of expression of the control marker in a population of subjects suffering from the inflammatory disease.

15. The method of 14, wherein the population of subjects suffering from the inflammatory disease has received at least one of the anti-TNF treatment and the anti-IL 17 treatment.

16. The method of 14, wherein the population of subjects suffering from the inflammatory disease has received the anti-TNF treatment and the anti-IL17 treatment.

17. The method of claim 1, wherein the control marker comprises a CXCL1 marker or a CXCL5 marker.

18. The method of claim 1, wherein the control marker comprises both a CXCL1 marker and a CXCL5 marker.

19. The method of claim 1, wherein the anti-TNF treatment comprises an anti-TNF binding protein.

20. The method of claim 19, wherein the anti-TNF binding protein comprises a fusion protein, an antibody, or antigen binding fragment thereof, that specifically binds to TNF or IL17.

21. The method of claim 19, wherein the anti-TNF binding protein comprises an anti-IL17 antibody, or antigen binding fragment thereof, and is a murine antibody, a human antibody, a humanized antibody, a bispecific antibody, a chimeric antibody, a Fab, a Fab′, a F(ab′)2, an ScFv, an SMIP, an affibody, an avimer, a versabody, a nanobody, a domain antibody, or an antigen binding fragment thereof.

22. The method of claim 21, wherein the anti-TNF antibody is an anti-TNFα antibody.

23. The method of claim 22, wherein the anti-TNF antibody comprises a human anti-TNF antibody.

24. The method of claim 23, wherein the anti-TNFα antibody comprises Adalimumab®, or an antigen binding fragment thereof.

25. The method of claim 22, wherein the anti-TNF antibody comprises a humanized anti-TNF antibody.

26. The method of claim 25, wherein the humanized anti-TNF antibody comprises infliximab, or an antigen binding fragment thereof.

27. The method of claim 19, wherein the anti-TNF binding protein comprises an anti-TNFα fusion protein.

28. The method of claim 27, wherein the anti-TNFα binding protein comprises etanercept, or an antigen binding fragment thereof.

29. The method of claim 1, wherein the anti-IL17 treatment comprises an anti-IL17 binding protein.

30. The method of claim 1, wherein the anti-IL17 treatment comprises an anti-IL17 antibody, or an antigen binding fragment thereof.

31. The method of claim 30, wherein the anti-IL17 antibody comprises a human antibody.

32. The method of claim 30, wherein the anti-IL17 antibody is selected from the group consisting of secukinumab, and RG7624, or an antigen binding fragment thereof.

33. The method of claim 30, wherein the anti-IL17 antibody comprises a humanized antibody.

34. The method of claim 33, wherein the anti-IL17 antibody is ixekizumab, 10F7, B6-17, or an antigen binding fragment thereof.

35. The method of claim 29, wherein the anti-IL17 binding protein comprises a fusion protein, an antibody, or antigen binding fragment thereof, that specifically binds to IL17.

36. The method of claim 30, wherein the anti-IL17 binding protein comprises an anti-IL17 antibody, or antigen binding fragment thereof, is a murine antibody, a human antibody, a humanized antibody, a bispecific antibody, a chimeric antibody, a Fab, a Fab′, a F(ab′)2, an ScFv, an SMIP, an affibody, an avimer, a versabody, a nanobody, a domain antibody, or an antigen binding fragment thereof.

37. The method of claim 1, wherein the anti-IL17 treatment comprises methotrexate, an analog thereof, or a pharmaceutically acceptable salt thereof.

38. The method of claim 1, wherein the anti-TNF treatment comprises methotrexate, an analog thereof, or a pharmaceutically acceptable salt thereof.

39. The method of claim 1, wherein both of the anti-TNF treatment and the anti-IL17 treatment comprises methotrexate, an analog thereof, or a pharmaceutically acceptable salt thereof.

40. The method of claim 1, wherein the combination therapy comprises the administration of a multispecific binding protein that binds at least one of TNF and IL17.

41. The method of claim 40, wherein the multispecific binding protein is selected from the group consisting of a dual variable domain immunoglobulin (DVD-Ig™) molecule, a half-body DVD-Ig (hDVD-Ig) molecule, a triple variable domain immunoglobulin (TVD-Ig) molecule, a receptor variable domain immunoglobulin (rDVD-Ig) molecule, a polyvalent DVD-Ig (pDVD-Ig) molecule), a monobody DVD-Ig (mDVD-Ig) molecule, a cross over (coDVD-Ig) molecule, a blood brain barrier (bbbDVD-Ig) molecule, a cleavable linker DVD-Ig (clDVD-Ig) molecule, and a redirected cytotoxicity DVD-Ig (rcDVD-Ig) molecule.

42. The method of claim 40, wherein the multispecific binding protein binds TNFα and IL17.

43. The method of claim 1, wherein the level of expression of at least one of the CXCL1 and the CXCL5 markers is determined.

44. The method of claim 1, wherein the level of expression of both the CXCL1 and the CXCL5 markers is determined.

45. The method of claim 44, wherein a lower level of expression of at least one of the CXCL1 and the CXCL5 marker(s) as compared to the level of expression of the control marker indicates that the combination therapy is effective.

46. The method of claim 44, wherein a lower level of expression of both the CXCL1 and the CXCL5 markers as compared to the level of expression of the control marker indicates that the combination therapy is effective.

47. The method of claim 1, wherein the subject has not been previously treated with a monotherapy comprising an anti-TNF treatment or a monotherapy comprising an anti-IL17 treatment.

48. The method of claim 1, wherein the combination therapy decreases the level of expression of at least one of the CXCL1 and the CXCL5 marker(s) to a greater extent than a monotherapy comprising an anti-TNF treatment.

49. The method of claim 1, wherein the combination therapy has a better clinical outcome or clinical endpoint than a monotherapy comprising an anti-TNF treatment.

50. The method of claim 1, wherein the subject does not respond to a monotherapy comprising an anti-TNF treatment.

51. The method of claim 1, wherein the combination therapy decreases the level of expression of at least one of the CXCL1 and the CXCL5 marker(s) to a greater extent than a monotherapy comprising an anti-IL17 treatment.

52. The method of claim 1, wherein the combination therapy has a better clinical outcome or clinical endpoint than a monotherapy comprising an anti-IL17 treatment.

53. The method of claim 1, wherein the subject does not respond to a monotherapy comprising an anti-IL17 treatment.

54. The method of claim 1, wherein the combination therapy decreases the level of expression of at least one of the CXCL1 and the CXCL5 marker(s) to a greater extent than both a monotherapy comprising an anti-TNF treatment and a monotherapy comprising an anti-IL17 treatment.

55. The method of claim 1, wherein the combination therapy has a better clinical outcome or clinical endpoint than both a monotherapy comprising an anti-TNF treatment and a monotherapy comprising an anti-IL17 treatment.

56. The method of claim 1, wherein the subject does not respond to either a monotherapy comprising an anti-TNF treatment or a monotherapy comprising an anti-IL17 treatment.

57. The method of claim 1, wherein the level of expression of the CXCL1 and/or the CXCL5 marker is determined at a nucleic acid level.

58. The method of claim 57, wherein the level of expression of the CXCL1 and/or the CXCL5 marker is determined by detecting RNA.

59. The method of claim 57, wherein the level of expression of the CXCL1 and/or the CXCL5 marker is determined by detecting mRNA, miRNA, or hnRNA.

60. The method of claim 57, wherein the level of expression of the CXCL1 and/or the CXCL5 marker is determined by detecting DNA.

61. The method of claim 57, wherein the level of expression of the CXCL1 and/or the CXCL5 marker is determined by detecting cDNA.

62. The method of claim 57, wherein the level of expression of the CXCL1 and/or the CXCL5 markers is determined by using a technique selected from the group consisting of a polymerase chain reaction (PCR) amplification reaction, reverse-transcriptase PCR analysis, quantitative reverse-transcriptase PCR analysis, Northern blot analysis, an RNAase protection assay, digital RNA detection/quantitation, and a combination or sub-combination thereof.

63. The method of claim 57, wherein determining the level of expression of at least one of the CXCL1 and the CXCL5 marker(s) in the sample comprises performing an immunoassay using an anti-CXCL1 and an anti-CXCL5 antibody.

64. The method of claim 1, wherein the CXCL1 and/or the CXCL5 marker comprise a protein.

65. The method of claim 64, wherein the protein is detected using a binding protein that binds at least one of the CXCL1 and the CXCL5 marker(s).

66. The method of claim 64, wherein the binding protein comprises an antibody, or antigen binding fragment thereof, that specifically binds to the protein.

67. The method of claim 66, wherein the antibody or antigen binding fragment thereof is selected from the group consisting of a murine antibody, a human antibody, a humanized antibody, a bispecific antibody, a chimeric antibody, a Fab, a Fab′, a F(ab′)₂, an scFv, an SMIP, an affibody, an avimer, a versabody, a nanobody, a domain antibody, and an antigen binding fragment thereof.

68. The method of claim 65, wherein the binding protein comprises a multispecific binding protein.

69. The method of claim 68, wherein the multispecific binding protein is selected from the group consisting of a dual variable domain immunoglobulin (DVD-Ig™) molecule, a half-body DVD-Ig (hDVD-Ig) molecule, a triple variable domain immunoglobulin (TVD-Ig) molecule, a receptor variable domain immunoglobulin (rDVD-Ig) molecule, a polyvalent DVD-Ig (pDVD-Ig) molecule), a monobody DVD-Ig (mDVD-Ig) molecule, a cross over (coDVD-Ig) molecule, a blood brain barrier (bbbDVD-Ig) molecule, a cleavable linker DVD-Ig (clDVD-Ig) molecule, and a redirected cytotoxicity DVD-Ig (rcDVD-Ig) molecule.

70. The method of claim 66, wherein the antibody or antigen binding fragment thereof comprises a label.

71. The method of claim 70, wherein the label is selected from the group consisting of a radio-label, a biotin-label, a chromophore, a fluorophore, and an enzyme.

72. The method of claim 1, wherein the level of expression of at least one of the CXCL1 and the CXCL5 marker(s) is determined by using a technique selected from the group consisting of an immunoassay, a western blot analysis, a radioimmunoassay, immunofluorimetry, immunoprecipitation, equilibrium dialysis, immunodiffusion, an electrochemiluminescence immunoassay (ECLIA), an ELISA assay, a polymerase chain reaction, an immunopolymerase chain reaction, and combinations or sub-combinations thereof.

73. The method of claim 72, wherein the immunoassay comprises a solution-based immunoassay selected from the group consisting of electrochemiluminescence, chemiluminescence, fluorogenic chemiluminescence, fluorescence polarization, and time-resolved fluorescence.

74. The method of claim 72, wherein the immunoassay comprises a sandwich immunoassay selected from the group consisting of electrochemiluminescence, chemiluminescence, and fluorogenic chemiluminescence.

75. The method of claim 1, wherein the sample comprises a fluid, or component thereof, obtained from the subject.

76. The method of claim 75, wherein the fluid is selected from the group consisting of blood, serum, synovial fluid, lymph, plasma, urine, amniotic fluid, aqueous humor, vitreous humor, bile, breast milk, cerebrospinal fluid, cerumen, chyle, cystic fluid, endolymph, feces, gastric acid, gastric juice, mucus, nipple aspirates, pericardial fluid, perilymph, peritoneal fluid, pleural fluid, pus, saliva, sebum, semen, sweat, serum, sputum, tears, vaginal secretions, and fluid collected from a biopsy.

77. The method of claim 1, wherein the sample comprises a tissue or cell, or component thereof, obtained from the subject.

78. The method of claim 1, wherein the subject is a human subject.

79. A kit for (i) determining whether a subject having an inflammatory disease will respond to treatment with a combination therapy comprising an anti-TNF treatment and an anti-IL17 treatment; (ii) monitoring the effectiveness of the combination therapy; (iii) selecting a subject for participation in a clinical trial for the combination therapy for the inflammatory disease; or (iv) identifying a combination therapy comprising an anti-TNF treatment and an anti-IL17 treatment for a subject having an inflammatory disease, the kit comprising:
reagents for determining a level of expression of at least one of a CXCL1 and a CXCL5 marker in a sample obtained from the subject;
a control marker; and
instructions for (i) determining whether the subject will respond to the combination therapy comprising; (ii) monitoring the effectiveness of the combination therapy; (iii) selecting a subject for participation in a clinical trial for the combination therapy; or (iv) identifying a combination therapy comprising an anti-TNF treatment and an anti-IL17 treatment for treating a subject having an inflammatory disease.

80. The kit of claim 79, wherein the combination therapy comprises a DVD-Ig molecule directed against TNF and IL17.

81. The kit of claim 80, wherein the DVD-Ig molecule is directed against TNFα and IL17.

82. The kit of claim 79, wherein the reagents for determining the level of expression of at least one of a CXCL1 and a CXCL5 marker comprise a probe for amplifying and detecting at least one of the CXCL1 and the CXCL5 marker.

83. The kit of claim 79, wherein the reagents for determining the level of expression of at least one of a CXCL1 and a CXCL5 marker comprise an antibody, or antigen binding fragment thereof.

84. The kit of claim 79, further comprising reagents for obtaining a biological sample from the subject.

85. A method of determining whether a subject having an inflammatory disease will respond to treatment with a combination therapy comprising an anti-TNFα antibody and an anti-IL17 antibody, the method comprising the steps of:
determining a level of expression of at least one of a CXCL1 and a CXCL5 marker in a sample obtained from the subject using a reagent that interacts with at least one of the CXCL1 and the CXCL5 marker(s) and transforms the sample such that at least one of the CXCL1 and the CXCL5 marker(s) can be detected; and
comparing the level of expression at least one of the CXCL1 and the CXCL5 marker(s) to the level of expression of a control marker,
wherein a higher level of expression of at least one of the CXCL1 and the CXCL5 markers, as compared to the level of expression of the control marker, and/or a lower level of expression of at least one of the CXCL1 and the CXCL5 markers after the combination therapy comprising the anti-TNF treatment and the anti-IL17 treatment has been administered, as compared to the level of expression of the control marker, indicates that the combination therapy will be effective in treating the subject.

86. The method of claim 85, wherein the reagent that interacts with at least one of the CXCL1 and the CXCL5 marker(s) is an anti-CXCL1 or an anti-CXCL5 antibody, or antigen binding fragment thereof.

87. The method of claim 86, wherein the anti-CXCL1 antibody is selected from the group consisting of EMD Millipore: AP1151-100UG; Everest Biotech: EB09637; Lifespan Biosciences: LS-B2843, LS-B2513, and LS-C108147, eBioscience: 50-7519-42 and 50-7519-41; AbD Serotec: AAM40B, AAM40, and AAR22B; Thermo Fisher Scientific, Inc.: PA1-32959, PA1-32924, and PA1-20861; Abbiotec: 251349, 12335-1-AP, and AP08852PU-N; NovaTeinBio: 63059; Abgent: AT1688a; Aviva Systems Biology: AVARP07032_P050, OASA08635, and OAEB00281; United States Biological: C8297-97A, C8298-01B, and C8298-01C; Creative Biomart: CAB-1005 MH, CAB-3086 MH, and CAB-115 MH; Novus Biologicals: NBP1-61297, NBP1-51486, and NBP1-19301; Abnova: H00002919-M01, H00002919-D01P, and H00002919-M03; Fitzgerald: 70R-10502; and ProSci: 31-057, 42-129, and 42-196.

88. The method of claim 86, wherein the anti-CXCL5 antibody is selected from the group consisting of Lifespan Biosciences: LS-B5529; AbD Serotec: AHP1279, AAM42, and AHP1279B; Proteintech Group: 10809-1-AP and PA1-29657; Biorbyt: orb13909 and orb13450; Acris Antibodies: AM31037PU-N, PP1003B2, and PP1003P1; NovaTeinBio: 63066, AT1694a, and AT1693a; Aiva Systems Biology: OASA07658, OASA08449, and OASA07657; United States Biological: C8297-98H1, C8297-98H, and E2275-07; Creative Biomart: CAB-5426 MH and CAB-5425 MH; Novus Biologicals: 33140002; and Abnova: H00006374-M05, H00006374-M03, and H00006374-B01.

89. The method of claim 85, wherein the reagent that interacts with at least one of the CXCL1 and CXCL5 marker(s) comprises a nucleic acid probe specific for at least one of the CXCL1 or CXCL5 marker(s).

90. The method of claim 86, wherein determining the level of expression of at least one of the CXCL1 and the CXCL5 marker(s) in the sample comprises performing an immunoassay using an anti-CXCL1 or an anti-CXCL5 antibody.

91. The method of claim 85, wherein determining the level of expression of at least one of the CXCL1 and the CXCL5 marker(s) in the sample comprises a novel combination of assays.

92. The method of claim 1, wherein the inflammatory disease comprises rheumatoid arthritis.

93. The method of claim 1, wherein the inflammatory disease comprises at least one of psoriasis, psoriatic arthritis, ankylosing spondylitis, juvenile idiopathic arthritis, Behcet's disease, spondyloarthritis, uveitis, and systemic lupus erythematosus.

94. The method of claim 1, wherein the marker comprises a gene product.

95. The method of claim 94, wherein the gene product comprises a protein or RNA.

96. A method of contraindicating a subject having an inflammatory disease from a combination therapy comprising an anti-TNF treatment and an anti-IL17 treatment, the method comprising the steps of determining that a subject exhibits a lower level of expression of at least one of a CXCL1 and a CXCL5 marker as compared to a level of expression of a control marker, or a normal range of laboratory values, wherein the lower level of expression of at least one of the CXCL1 and the CXCL5 markers, as compared to the level of expression of the control marker, indicates that the subject is not contraindicated for a combination therapy comprising an anti-TNF treatment and an anti-IL17 treatment.
